(19) United States
(12) Patent Application Publication
(10) Pub. No.: US 2005/0142141 A1
(43) Pub. Date: Jun. 30, 2005

(54) Delivery of enzymes to the brain

(76) Inventor: William M. Pardridge, Pacific Palisades, CA (US)

Correspondence Address: David J. Oldenkamp, Esq.;Shapiro & Dupont LLP, Suite 700, 233 Wilshire Boulevard, Santa Monica, CA, 90401, US

(21) Appl. No.: 11/061,956

(22) Filed: Feb. 17, 2005

Related U.S. Application Data
(63) Continuation-in-part of application No. 10/307,276, filed on Nov. 27, 2002.

(51) Int. Cl. A61K 39/395; A61K 38/47

(52) U.S. Cl. 424/178.1; 424/94.61; 435/188.5

(57) ABSTRACT

Delivery of large enzymes to the brain via transport across the blood-brain barrier (BBB) utilizing conjugates, or fusion proteins, which are composed of a therapeutic enzyme and a BBB targeting agent (molecular Trojan horse). The enzyme is missing in the brain, and does not cross the BBB. The molecular Trojan horse is a receptor-specific endogenous peptide, or peptidomimetic monoclonal antibody (MAb), that undergoes receptor-mediated transport across the BBB, thereby carrying into brain the attached enzyme.

### BACKGROUND OF THE INVENTION

This is a continuation-in-part of co-pending application Ser. No. 10/307,276, which was filed on Nov. 27, 2002, and which is assigned to the same assignee as the present application.

1. Field of the Invention

The present invention relates generally to the delivery of pharmaceutical agents from the blood stream to the human brain and other organs or tissues that express the human insulin receptor. More particularly, the present invention involves the development of “humanized” monoclonal antibodies (MAb) that may be attached to pharmaceutical agents to form compounds that are able to readily bind to the human insulin receptor (HIR). The compounds are able to cross the human blood brain barrier (BBB) by way of insulin receptors located on the brain capillary endothelium. Once across the BBB, the humanized monoclonal antibody/pharmaceutical agent compounds are also capable of undergoing receptor mediated endocytosis into brain cells via insulin receptors located on the brain cells.

In addition, the present invention relates to the delivery of enzymes to the brain via transport across the blood-brain barrier (BBB). In particular, the invention relates to the production of conjugates, or fusion proteins, which are composed of a therapeutic enzyme and a molecular Trojan horse. The therapeutic enzyme is missing in the brain, and does not cross the BBB. The molecular Trojan horse is a receptor-specific endogenous peptide, or peptidomimetic monoclonal antibody (MAb), that undergoes receptor-mediated transport across the BBB, thereby carrying into brain the attached enzyme that the brain is missing.

2. Description of Related Art

The publications and other reference materials referred to herein to describe the background of the invention and to provide additional detail regarding its practice are hereby incorporated by reference. For convenience, the reference materials are identified by author and date and grouped in the appended bibliography.

The BBB is a system-wide membrane barrier that prevents the brain uptake of circulating drugs, protein therapeutics, antisense drugs, and gene medicines. Drugs or genes can be delivered to the human brain for the treatment of serious brain disease either (a) by injecting the drug or gene directly into the brain, thus bypassing the BBB, or (b) by injecting the drug or gene into the bloodstream so that the drug or gene enters the brain via the transvascular route across the BBB. With intra-cerebral administration of the drug, it is necessary to drill a hole in the head and perform a procedure called craniotomy. In addition to being expensive and highly invasive, this craniotomy based drug delivery to the brain approach is ineffective, because the drug or gene is only delivered to a tiny volume of the brain at the tip of the injection needle. The only way the drug or gene can be distributed widely in the brain is the transvascular route following injection into the bloodstream. However, this latter approach requires the ability to undergo transport across the BBB. The BBB has proven to be a very difficult and stubborn barrier to traverse safely.

Prior work has shown that drugs or gene medicines can be ferried across the BBB using molecular Trojan horses that bind to BBB receptor/transport systems. These Trojan horses may be modified proteins, endogenous peptides, or peptidomimetic monoclonal antibodies (MAb's). For example, HIR MAb 83-14 is a murine MAb that binds to the human insulin receptor (HIR). This binding triggers transport across the BBB of MAb 83-14 (Pardridge et al, 1995), and any drug or gene payload attached to the MAb (Wu et al., 1997).

The use of molecular Trojan horses to ferry drugs or genes across the BBB is described in U.S. Pat. Nos. 4,801,575 and 6,372,250. The linking of drugs to MAb transport vectors is facilitated with use of avidin-biotin technology. In this approach, the drug or protein therapeutic is monobiotinylated and bound to a conjugate of the antibody vector and avidin or streptavidin. The use of avidin-biotin technology to facilitate linking of drugs to antibody-based transport vectors is described in U.S. Pat. No. 6,287,792. Fusion proteins have also been used where a drug is genetically fused to the MAb transport vector.

HIRMAb 83-14 has been shown to rapidly undergo transport across the BBB of a living Rhesus monkey, and to bind avidly to isolated human brain capillaries, which are the anatomical substrate of the human BBB (see Pardridge et al., 1995). In either case, the activity of the HIRMAb 83-14 with respect to binding and transport at the primate or human BBB is more than 10-fold greater than the binding or transport of other peptidomimetic MAb's that may target other BBB receptors such as the transferrin receptor (Pardridge, 1997). To date, HIRMAb 83-14 is the most active BBB transport vector known (Pardridge, 1997). On this basis, the HIRMAb 83-14 has proven to be a very useful agent for the delivery of drugs to the primate brain in vivo, and would also be highly active for brain drug or gene delivery to the brain in humans.

HIRMAb 83-14 cannot be used in humans because this mouse protein will be immunogenic. Genetically engineered forms of HIRMAb 83-14 might be used in humans in either the form of a chimeric antibody or a genetically engineered “humanized” HIRMAb. However, in order to perform the genetic engineering and production of either a chimeric or a humanized antibody, it is necessary to first clone the variable region of the antibody heavy chain (VH) and the variable region of the antibody light chain (VL). Following cloning of the VH and VL genes, the genes must be sequenced and the amino acid sequence deduced from the nucleotide sequence. With this amino acid sequence, using technologies known to those skilled in the art (Foote et al., 1992), it may be possible to perform humanization of the murine HIRMAb 83-14. However, HIRMAb 83-14 may lose biological activity following the humanization (Pichla et al., 1997). Therefore, it is uncertain as to whether the murine HIRMAb can be humanized with retention of biological activity.

A chimeric form of the HIRMAb 83-14 has been genetically engineered, and the chimeric antibody binds to the HIR and is transported into the primate brain (Coloma et al., 2000). However, a chimeric antibody retains the entire mouse FR for both the VH and the VL, and because of this, chimeric antibodies are still immunogenic in humans (Bruggemann et al., 1989). In contrast to the chimeric antibody, a humanized antibody would use the human FR amino acid sequences for both the VH and the VL and retain only the murine amino acids for the 3 complementarity determining regions (CDRs) of the VH and 3 CDRs of the VL. Not all murine MAb's can be humanized, because there is a loss of biological activity when the murine FR's are replaced by human FR sequences (Pichla et al., 1997). The biological activity of the antibody can be restored by substituting back certain mouse FR amino acids (see U.S. Pat. No. 5,585,089). Nevertheless, even with FR amino acid back-substitution, certain antibodies cannot be humanized with retention of biological activity (Pichla et al., 1997). Therefore, there is no certainty that the murine HIRMAb 83-14 can be humanized even once the key murine CDR and FR amino acid sequences are known.

There are over 40 lysosomal storage disorders, which are inborn errors of metabolism caused by an inherited mutation in a specific gene, which encodes for a lysosomal enzyme (Kaye, 2001). The lysosomal enzyme normally degrades accumulated by-products in the cell, such as glycosaminoglycans, glycolipids, and other lysosomal storage products. More than half of the lysosomal storage disorders affect the brain, often times very adversely (Cheng and Smith, 2003). The lysosomal storage diseases are treated with Enzyme Replacement Therapy or ERT. In ERT, the patient is typically given an intravenous infusion of the recombinant enzyme at periodic intervals. The recombinant enzyme is produced with standard biotechnology and genetic engineering techniques following the cloning and sequencing of the cDNA encoding the lysosomal enzyme. Virtually all of the lysosomal enzyme genes have been cloned (Table 4), and all of the missing enzymes could be produced for human treatment using ERT. Table 4 gives a partial list of lysosomal storage disorders affecting the brain. The missing enzyme for each of these diseases could be produced for human therapy, since all of the genes have been isolated and cloned. The GenBank accession number given in Table 4 allows those skilled in the art to obtain the nucleotide sequence of the full length cDNA encoding each enzyme with standards methods, such as the polymerase chain reaction (PCR) method, and mass produce the enzyme. However, ERT of brain disorders has not been realized, because of the Achilles heel of the field—the enzymes once introduced into the bloodstream cannot enter the brain (Kaye, 2001).

The limiting factor in the ERT of the lysosomal storage disorders is the failure of any of the enzymes to undergo transport across the brain capillary endothelial wall, which forms the BBB in vivo (Pardridge, 2001). Indeed, the BBB is the limiting factor in virtually all brain drug development programs, since >98% of all small molecule drugs do not cross the BBB, and ˜100% of all large molecule drugs, such as enzymes, do not cross the BBB (Pardridge, 2001). Because of the BBB problem, attempts have been made to deliver the missing enzyme via a hole drilled in the head (Kakkis et al, 2004). In this approach a catheter is inserted into the internal ventricular compartment of the brain, which houses the cerebrospinal fluid (CSF). However, this ‘trans-cranial’ brain drug delivery strategy is invasive, expensive, and ineffective. It is ineffective because, CSF is normally pumped out of the brain every 4 hours in humans (Pardridge, 2001). This bulk flow of CSF substance back to the peripheral bloodstream is rapid compared to the slow diffusion of the drug, or enzyme from the CSF compartment down into brain tissue. Consequently, drug or enzyme that is introduced into the CSF compartment is only delivered to the surface of the brain, as demonstrated by Kakkis et al (2004), despite the infusion into the dog brain of volumes nearly equal to the entire CSF volume. The problem in delivery of enzyme to only the meningeal surface of the brain is that the lysosomal storage products accumulate in all cells of the brain. Therefore, an effective therapeutic strategy requires that the missing enzyme be delivered to virtually all cells in the brain.

The only way that a drug, or enzyme, can be delivered to all cells in the brain is via a trans-vascular, i.e., trans-BBB drug delivery approach (Pardridge, 2001). The brain is richly perfused with billions of tiny capillaries that form the BBB. The human brain has 400 miles of capillaries, which form a total surface area of 20 m². The distance between capillaries in the brain is about 50 μm. Therefore, virtually every neuron in the brain is perfused by its own blood vessel capillary. Once a drug, or enzyme, is delivered across the BBB, the pharmaceutical is delivered to the ‘doorstep’ of every cell in the brain (Pardridge, 2002).

The traditional approach to delivery of drugs across the BBB is called ‘BBB disruption.’ In this approach, a noxious agent or chemical is infused into the carotid artery, and this chemical causes a transient disruption of the BBB followed a short time later by closure of the BBB. However, BBB disruption allows all components of the blood or plasma to enter the brain, and blood proteins are toxic to brain cells. Chronic neuropathologic changes take place in the brain following BBB disruption (Pardridge, 2001). Accordingly, this approach has not gained widespread clinical acceptance.

Drugs, or enzymes, may be delivered to the brain without disrupting the BBB by taking advantage of the many endogenous transport systems that are expressed within the BBB. Glucose is needed on a second-to-second basis by the brain. Glucose is too water soluble to normally cross the BBB via free diffusion. However, glucose readily penetrates the BBB owing to its affinity for the endogenous BBB glucose transporter, which is a product of the GLUT1 gene (Pardridge et al, 1990). Similarly, the brain needs new neutral amino acids from the blood for protein synthesis, and circulating amino acids gain access to the brain via transport across the endogenous BBB large neutral amino acid transporter, which is a product of the LAT1 gene (Boado et al, 1999). In addition to small molecules, circulating peptides may also gain access to the brain via receptor-mediated transport (RMT) across the BBB. Circulating insulin enters brain via the endogenous BBB insulin receptor (IR), which is a product of the INSR gene (Pardridge et al, 1985). Similarly, blood-borne transferrin (Tf) enters brain via the endogenous BBB Tf receptor (TfR), which is a product of the TRFR gene (Pardridge et al, 1987). Either insulin or Tf could be used as molecular Trojan horses to ferry across the BBB any attached drug or enzyme, as taught in U.S. Pat. No. 4,801,575. The attachment of a drug or enzyme, that is not normally transported across the BBB, to a transportable peptide, such as insulin or Tf, results in the formation of a chimeric peptide. Chimeric peptides are bi-functional proteins, which can both (a) undergo receptor-mediated transport across the BBB via an endogenous peptide receptor, and (b) exert a pharmacological effect in brain, once the non-transportable therapeutic is delivered across the BBB.

In addition to endogenous peptides, antibodies to peptide receptors, such as an antibody to the transferrin receptor (Domingo and Trowbridge, 1985), an antibody to the insulin receptor (Schechter et al, 1982), or an antibody to the low density lipoprotein receptor (Beisiegel et al, 1981), may mimic the action of the endogenous peptide, and bind a target receptor, which then triggers a biological effect that mimics that of the endogenous peptide. Such MAb's are designated peptidomimetic antibodies. Anti-TfR MAb's or anti-IR MAb's bind BBB receptors, which triggers transport of the MAb across the BBB (Pardridge et al, 1991; Pardridge et al, 1995). Therefore, either the endogenous peptide, or a peptidomimetic MAb, may be used as a molecular Trojan horse to ferry drugs across the BBB.

In the case of enzyme delivery to the brain, it is necessary to circumvent a second barrier once the BBB is traversed. The enzyme must be targeted to the lysosome, and lysosomal enzymes carry motifs that target the enzyme to the lysosome (Arighi et al, 2004). However, the enzyme must first be transported across the ‘second barrier,’ which is the brain cell membrane (BCM). The 2 barriers in brain, the BBB and the BCM are depicted in FIG. 6. The BCM expresses both the TfR and the IR (Pardridge, 2001). Therefore, a TfR- or IR-specific MAb, acting as a molecular Trojan horse (TH, FIG. 6) could deliver the attached enzyme (E, FIG. 6) from blood to the intracellular space of brain, as shown in FIG. 6. This is accomplished by the sequential receptor-mediated transcytosis across the BBB followed by receptor-mediated endocytosis across the BCM. Once inside brain cells, the enzyme is targeted to lysosomes, where accumulated substrate (S, FIG. 6) is converted into low molecular weight product (P, FIG. 6).

The delivery of a large molecular weight (MW) enzyme to the brain that is depicted in FIG. 6 mimics a process that has been previously demonstrated for a range of peptide drugs, such as vasoactive intestinal peptide (VIP), which has a MW of about 5000 Daltons (Wu et al, 1996), to recombinant CD4, which has a MW of about 40,000 Daltons (Pardridge et al, 1992). However, many of the missing lysosomal enzymes have molecular weights of 50,000 to 100,000 Daltons; the MW of the individual enzymes can be found by accessing information with the GenBank accession number (Table 4). For example, β-glucuronidase (GUSB), following glycosylation, has a MW of about 85,000 Daltons (Gehrmann et al, 1994). Moreover, this enzyme, similar to μ-galactosidase, forms a homo-tetramer, and the MW of tetramer is 390,000 Daltons (Gehrmann et al, 1994). It is not known if BBB molecular Trojan horses can carry across the BBB therapeutic agents of this large size and with such high MW. An enzyme of 390,000 Daltons has a size nearly 3-fold greater than a 150,000 Dalton receptor-specific MAb, acting as a BBB molecular Trojan horse.

### SUMMARY OF THE INVENTION

In accordance with the present invention, it was discovered that the murine HIRMAb 83-14 antibody can be humanized to provide a biologically active humanized insulin receptor (HIR) antibody that may be used in combination with drugs and diagnostic agents to treat human beings in vivo. The HIR antibody may be conjugated to the drug or diagnostic agent using avidin-biotin conjugation or the HIR antibody/drug combination may be prepared as a fusion protein using genetic engineering techniques. The HIR antibody is especially well suited for delivering neuropharmaceutical agents to the human brain across the BBB. The humanized character of the HIR antibody significantly reduces immunogenic reactions in humans.

The humanized murine antibody of the present invention is capable of binding to the HIR and includes a heavy chain (HC) of amino acids and a light chain (LC) of amino acids which both include variable and constant regions. The variable regions of the HC and LC include complementarity determining regions (CDRs) that are interspersed between framework regions (FRs).

The HC includes a first CDR located at the amino end of the variable region, a third CDR located at the carboxyl end of the HC variable region and a second CDR located between said first and third CDRs. The amino acid sequences for the first CDR, the second CDR, and the third CDR are SEQ. ID. NOS. 31, 33 and 35, respectively, and combined equivalents thereof. The HC framework regions include a first FR located adjacent to the amino end of the first CDR, a second FR located between said first and second CDRs, a third FR located between said second and third CDRs and a fourth FR located adjacent to the carboxyl end of said third CDR. In accordance with the present invention, the four FRs of the HC are humanized such that the overall antibody retains biological activity with respect to the HIR and is not immunogenic in humans.

The LC also includes a first CDR located at the amino end of the variable region, a third CDR located at the carboxyl end of the variable region and a second CDR located between said first and third CDRs. The amino acid sequences for the first CDR, the second CDR, and the third CDR are SEQ. ID. NOS. 38, 40, and 42, respectively, and combined equivalents thereof. The LC framework regions include a first FR located adjacent to the amino end of said first CDR, a second FR located between said first and second CDRs, a third FR located between said second and third CDRs and a fourth FR located adjacent to the carboxyl end of said third CDR. Pursuant to the present invention, the four FRs of the LC are humanized such that the overall antibody retains biological activity with respect to the HIR and has minimal immunogenicity in humans.

The constant regions of the murine antibody are also modified to minimize immunogenicity in humans. The murine HC constant region is replaced with the HC constant region from a human immunoglobulin such as IgG1. The murine LC constant region is replaced with a constant region from the LC of a human immunoglobulin such as a kappa (κ) LC constant region. Replacement of the murine HC and LC constant regions with human constant regions was found to not adversely affect the biological activity of the humanized antibody with respect to HIR binding.

The present invention not only covers the humanized murine antibodies themselves, but also covers pharmaceutical compositions that are composed of the humanized antibody linked to a drug or diagnostic agent. The humanized antibody is effective in delivering the drug or diagnostic agent to the HIR in vivo to provide transport across the BBB and/or endocytosis into cells via the HIR. The compositions are especially well suited for intra venous (iv) injection into humans for delivery of neuropharmaceutical agents to the brain.

As another feature, the present invention is based on the unexpected finding that BBB molecular Trojan horses (such as the above-described receptor-specific Mab) can, in fact, deliver a high MW enzyme across the BBB to generate the desired pharmacological effect, which is an increase in brain enzyme activity. The use of Trojan horses to deliver lysosomal enzymes and other high molecular weight enzymes to the brain is useful in treating a wide variety of lysosomal storage disorders and other conditions where the enzyme being delivered is missing from the brain cell.

The above discussed and many other features and attendant advantages of the present invention will become better understood by reference to the detailed description when taken in conjunction with the accompanying drawings.

### BRIEF DESCRIPTION OF THE DRAWINGS

FIGS. 1A and 1B shows the nucleotide sequence for the murine VH (SEQ. ID. NO. 1) and murine VL (SEQ. ID. NO. 2) and deduced amino acid sequence of the murine VH (SEQ. ID. NO. 3) and the murine VL (SEQ. ID. NO. 4), which shows the 3 framework (FR) regions and the 4 complementarity determining regions (CDRs) of both the heavy chain (HC) and the light chain (LC) of the 83-14 murine HIRMAb. The amino acids denoted by an asterisk (*) were confirmed by amino acid sequencing of either the intact murine LC or tryptic peptides of the intact murine HC; for amino acid sequencing, the intact murine HC or LC were purified from gels following purification of the intact murine IgG from the hybridoma conditioned medium.

FIGS. 2A and 2B graphically show the results of a radio-receptor assay on isolated human brain capillaries that were obtained with a mechanical homogenization procedure from human autopsy brain. These capillaries were incubated with [¹²⁵I]-labeled chimeric HIRMAb (Coloma et al., 2000) (FIG. 2A) or [¹²⁵I]-version 5 humanized HIRMAb (FIG. 2B). The data show that both antibodies bind equally well to human brain capillaries, which form the anatomical basis of the BBB in humans.

FIG. 3 shows the brain scan of a Rhesus monkey treated with a humanized monoclonal antibody in accordance with the present invention. The [¹²⁵I]-labeled version HIRMAb was injected intravenously in an anesthetized rhesus monkey, and the animal was euthanized 120 minutes later. The brain was rapidly removed and cut into coronal hemispheric slabs, which were immediately frozen. Cryostat sections (20 μm) were cut and exposed to x-ray film. The film was scanned to yield the image shown in FIG. 3. This image shows the clear demarcations between the gray matter and white matter of the primate brain. Owing to the higher vascular density in gray matter, there is a greater uptake of the humanized HIRMAb, relative to white matter.

FIG. 4 shows a comparison of the amino acid sequence for the 3 FRs and 3 CDRs of both the heavy chain and the light chain for the following: (a) the version 5 humanized HIRMAb, (v) the original murine 83-14 HIRMAb, and (c) the VH of the B43 human IgG or the VL of the REI human IgG.

FIG. 5 shows the amino acid sequence of a fusion protein of human □-L-iduronidase (IDUA) (SEQ. ID. NO. 48), which is fused to the carboxyl terminus of the heavy chain (HC) of the humanized monoclonal antibody to the human insulin receptor (HIRMAb). The HC is comprised of a variable region (VH) and a constant region (CH); the CH is further comprised of 3 sub-regions, CH1 (SEQ. ID. NO. 44), CH2 (SEQ. ID. NO. 45), and CH3 (SEQ. ID NO. 46); the CH1 and CH2 regions are connected by a 12 amino acid hinge region (SEQ. ID. NO. 47). The VH is comprised of 4 framework regions (FR1=SEQ. ID. NO. 30; FR2=SEQ. ID. NO. 32; FR3=SEQ. ID. NO. 34; and FR4=SEQ. ID. NO. 36) and 3 complementarity determining regions (CDR) (CDR1=SEQ. ID. NO. 31; CDR2=SEQ. ID. NO. 33; and CDR3=SEQ. ID. NO. 35). The amino acid sequence shown for the CH is well known in existing databases and corresponds to the CH sequence of human IgG1. There is a single N-linked glycosylation site on the asparagine (N) residue within the CH2 region of the CH, and there are 6 potential N-linked glycosylation sites within the IDUA sequence, as indicated by the underline.

FIG. 6 depicts enzyme delivery to brain. A chimeric peptide is formed by fusing a non-transportable enzyme, E, to a BBB molecular Trojan horse, TH. The TH binds a specific receptor on the BBB, and this enables transport across the BBB. In the example shown here, the TH is a MAb to the BBB insulin receptor (IR). The E/TH chimeric peptide then binds the IR on the brain cell plasma membrane via receptor-mediated endocytosis. Once inside brain cells, the enzyme part of the chimeric peptide may then degrade lysosomal storage polymers, or substrate (S), into low molecular weight products (P). Without attachment to the Trojan horse, the enzyme cannot cross the BBB and is not pharmacologically active in brain following systemic administration. The Trojan horse could also target the transferrin receptor (TfR), or other BBB receptor systems.

FIG. 7 depicts conjugate synthesis. (A) Reaction I: Thiolation of the 8D3 TfRMAb with Traut's reagent is performed in parallel with the activation of recombinant streptavidin (SA) with S-SMPB. The thiolated 8D3 MAb and activated SA are conjugated to form a stable thiol-ether linkage between the 8D3 MAb and SA. Reaction II: Bacterial β-galactosidase is mono-biotinylated with sulfo-NHS-LC-LC-biotin. The double LC linker provides a 14-atom spacer between the biotin moiety and the epsilon-amino group of surface lysine residues on the enzyme. Reaction III: The β-galactosidase-8D3 conjugate is formed upon mixing the mono-biotinylated β-galactosidase (β-gal-LC-LC-biotin) and the 8D3-SA conjugate. (B) SDS-PAGE of molecular weight standards (left lane) and β-galactosidase (right lane). The size of the molecular weight standards is shown in the figure. The β-galactosidase migrates at a molecular weight of 116 kDa. (C) The β-galactosidase enzyme activity is unchanged following conjugation to the 8D3 monoclonal antibody. Data are mean±SE (n=3).

FIG. 8 depicts the results of a low dose injection study. Percent of injected dose (ID) per gram tissue is shown for mouse liver, spleen, kidney, heart and brain (inset) at 60 min after an intravenous (IV) injection of a low dose (15 ug/mouse) of β-galactosidase in either the unconjugated form (closed bars) or as a conjugate with the 8D3 TfRMAb (open bars). Data are mean±SE (n=3). The injected dose per gram organ was computed from the specific activity of the injected enzyme or enzyme-8D3 conjugate (mU/ug) and the injected dose of enzyme (ug). The endogenous β-galactosidase enzyme activity, measured in organs removed from un-injected animals, was subtracted for each organ.

FIG. 9 shows the results of a high dose injection study. Percent of injected dose (ID) per gram tissue is shown for mouse liver, spleen, kidney, heart and brain (inset) at 60 min after an IV injection of a high dose (150 ug/mouse) of β-galactosidase in either the unconjugated form (closed bars) or as a conjugate with the 8D3 TfRMAb (open bars). Data are mean±SE (n=3). The injected dose per gram organ was computed from the specific activity of the injected enzyme or enzyme-8D3 conjugate (mU/ug) and the injected dose of enzyme (ug). The endogenous β-galactosidase enzyme activity (Table 1) was subtracted for each organ.

FIG. 10 shows brain histochemistry. Mouse brain was saline flushed and perfusion fixed at 60 minutes following intravenous injection of a maximal dose (300 ug/mouse) of either the β-galactosidase-8D3 conjugate (panels A and B) or the unconjugated β-galactosidase (panel C). The magnification bar in panel A is 48 microns. The magnification bar in panel B is 180 microns. The magnification of panels B and C are identical.

FIG. 11 depicts the results of tests using the capillary depletion method. β-galactosidase enzyme activity in the brain homogenate and the post-vascular supernatant at 60 minutes following intravenous injection of the 150 ug/mouse high dose of the β-galactosidase/8D3 conjugate. Data are mean±SE (n=3 mice). The post-vascular supernatant and the homogenate were separated with the capillary depletion technique.

### DETAILED DESCRIPTION OF THE INVENTION

The present invention involves the humanization of the murine monoclonal antibody identified as MAb 83-14 so that it can be used in vivo in humans. As previously mentioned, MAb 83-14 has a high affinity for the human insulin receptor at the human or rhesus monkey blood-brain barrier (Pardridge, et al. 1995) and is a candidate for use as a Trojan horse to transport neuropharmaceutical agents across the BBB. As used herein, the term “pharmaceutical agents” is intended to include any drug, gene or chemical that is used to treat or diagnose disease in humans. The term “neuropharmaceutical agent” covers pharmaceutical agents that are used to treat brain disease. The present humanized antibody Trojan horses are especially well suited for transporting neuropharmaceutical agents from the blood stream to the brain across the BBB.

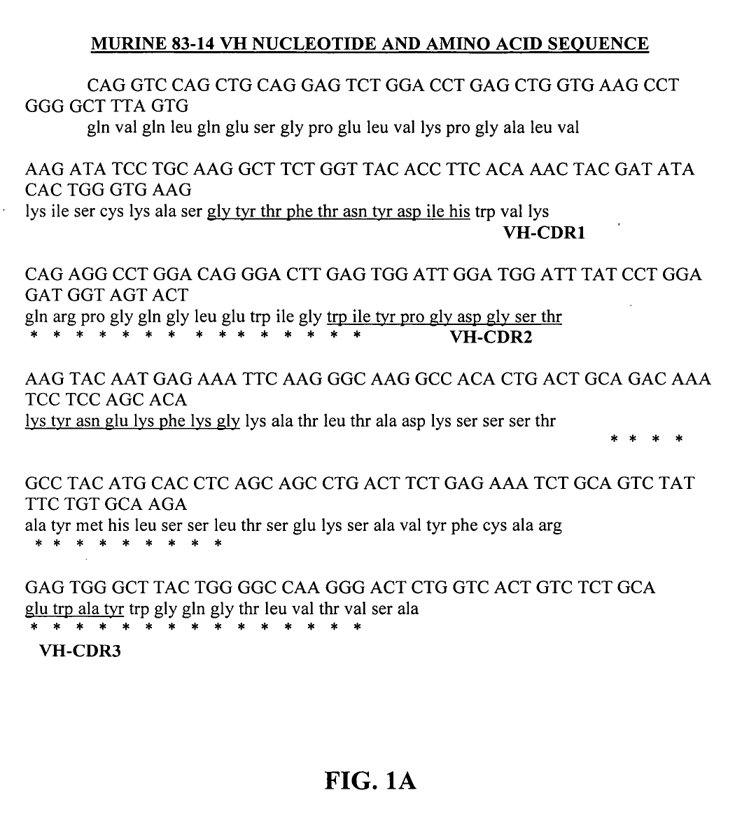

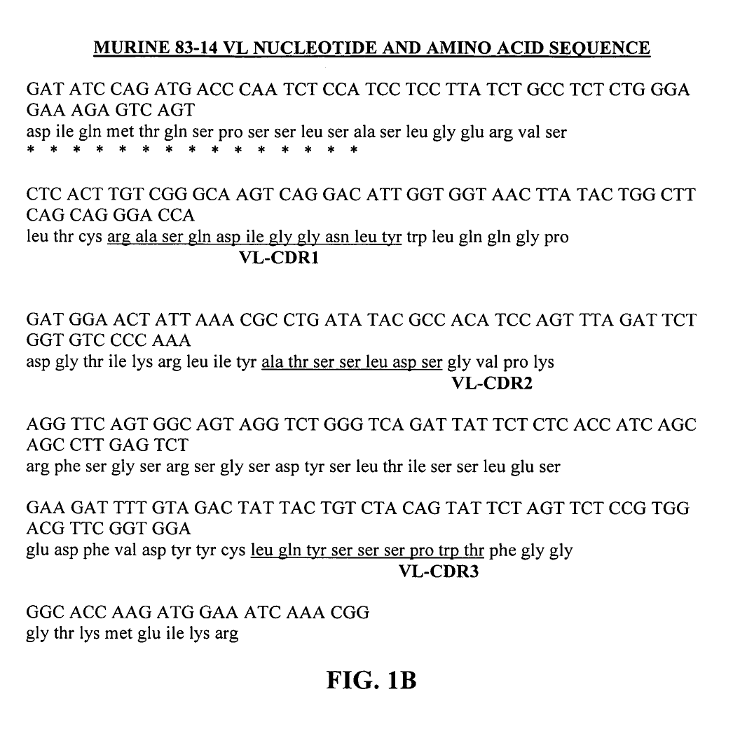

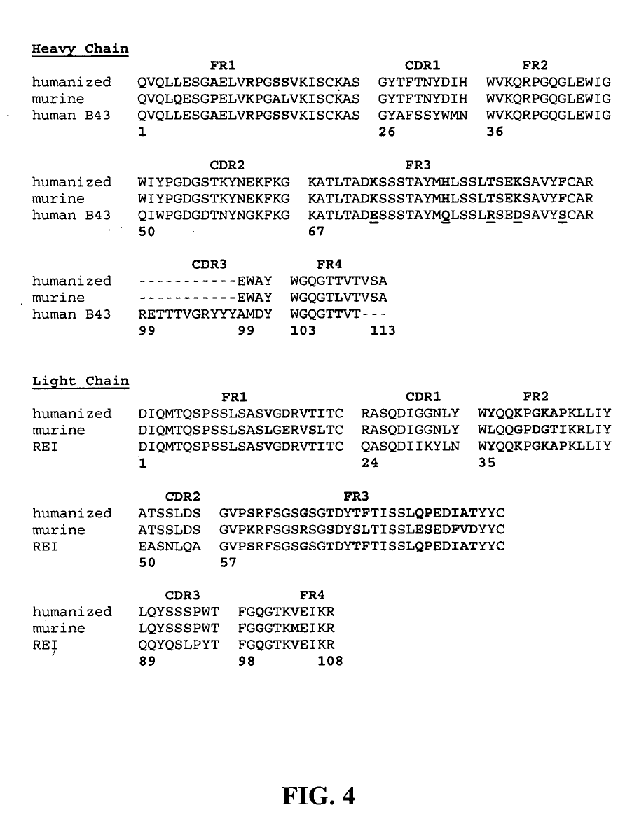

The complete amino acid sequence for the variable region of the HC and LC of murine Mab 83-14 was determined as described in Example 1. The nucleotide sequence for the gene that expresses the murine VH (SEQ. ID. NO. 1) and the murine VL (SEQ. ID. NO. 2) is set forth in FIG. 1. The amino acid sequence for the murine VH (SEQ. ID. NO. 3) and murine VL (SEQ. ID. NO. 4) is also set forth in FIG. 1. The amino acid sequences for the variable regions of the murine MAb 83-14 VH and VL are also set forth in FIG. 4 (SEQ. ID. NOS. 3 AND 4, respectively). The humanized murine antibodies of the present invention are prepared by modifying the amino acid sequences of the variable regions of the murine antibody to more closely resemble human antibody without destroying the ability of the antibody to strongly bind to the HIR. In addition, the humanized antibody includes constant regions that also correspond to human antibody.

The humanized murine antibodies include a heavy chain of amino acids (HC) that is composed of a constant region (CH) and a variable region (VH). The variable region of the HC has an amino end and a carboxyl end and includes three CDRs interspersed between four FRs. The first CDR (CDR1) is located towards the amino end of the VH with the third CDR (CDR3) being located towards the carboxyl end of the HC. The amino acid sequences for murine MAb 83-14 HC CDR1, CDR2, and CDR3 are set forth in SEQ. ID. NOS. 31, 33 and 35, respectively. Since the HC CDRs are essential for antibody binding to the HIR, it is preferred that the humanized antibodies have HC CDRs with amino acid sequences that are identical to SEQ. ID. NOS. 31, 33 and 35. However, the humanized antibodies may include CDRs in the HC that have amino acid sequences which are “individually equivalent” to SEQ. ID. NOS. 31, 33 and 35. “Individually equivalent” amino acid sequences are those that have at least 75 percent sequence identity and which do not adversely affect the binding of the antibody to the HIR. Preferably, individually equivalent amino acid sequences will have at least 85 percent sequence identity with SEQ. ID. NOS. 31, 33 or 35. Even more preferred are individually equivalent amino acid sequences having at least 95 percent sequence identity.

The three VH CDR amino acid sequences may also be viewed as a combined group of amino acid sequences (VH CDR1, VH CDR2 and VH CDR3). The present invention also covers equivalents of the combined group of VH CDR sequences. Such “combined equivalents” are those that have at least 75 percent sequence identity with the combined amino acid sequences SEQ. ID. NOS. 31, 32 and 35 and which do not adversely affect the binding of the antibody to the HIR. Preferably, combined equivalent amino acid sequences will have at least 85 percent sequence identity with the combined sequences found in SEQ. ID. NOS. 31, 33 and 35. Even more preferred are combined equivalent amino acid sequences that have at least 95 percent sequence identity with the combined amino acid sequences (SEQ. ID. NOS. 31, 33 and 35).

It is preferred that the VH CDR amino acid sequences meet both the individual equivalency and combined equivalency requirements set forth above. However, there are certain situations, especially for the shorter CDRs, where one or more of the CDRs may not meet the criteria for individual equivalence even though the criteria for combined equivalence is met. In such situations, the individual equivalency requirements are waived provided that the combined equivalency requirements are met. For example, VH CDR3 (SEQ. ID. NO. 35) is only 4 amino acids long. If two amino acids are changed, then the individual sequence identity is only 50% which is below the 75% floor for individual equivalence set forth above. However, this particular sequence is still suitable for use as part of a combined equivalent VH CDR group provided that the sequence identity of the combined CDR1, CDR2 and CDR3 sequences meet the group equivalency requirements.

The humanized murine antibodies also include a light chain (LC) of amino acids that is composed of a constant region (CL) and a variable region (VL). The variable region of the LC has an amino end and a carboxyl end and includes three CDRs interspersed between four FRs. The first CDR (CDR1) is located towards the amino end of the VL with the third CDR (CDR3) being located towards the carboxyl end of the VL. The amino acid sequences for murine MAb 83-14 LC CDR1, CDR2, and CDR3 are set forth in SEQ. ID. NOS. 38, 40 and 42, respectively. Since the VL CDRs are also important for antibody binding to the HIR, it is preferred that the humanized antibodies have LC CDRs with amino acid sequences that are identical to SEQ. ID. NOS. 38, 40 and 42. However, the humanized antibodies may include CDRs in the VL that have amino acid sequences which are “individually equivalent” to SEQ. ID. NOS. 38, 40 or 42. “Individually equivalent” amino acid sequences are those that have at least 75 percent sequence identity and which do not adversely affect the binding of the antibody to the HIR. Preferably, individually equivalent amino acid sequences will have at least 85 percent sequence identity with SEQ. ID. NOS. 38, 40 or 42. Even more preferred are individually equivalent amino acid sequences having at least 95 percent sequence identity.

The three VL CDR amino acid sequences may also be viewed as a combined group of amino acid sequences (VL CDR1, VL CDR2 and VL CDR3). The present invention also covers equivalents of the combined group of VL CDR sequences. Such “combined equivalents” are those that have at least 75 percent sequence identity with the combined amino acid sequences SEQ. ID. NOS. 38, 40 and 42 and which do not adversely affect the binding of the antibody to the HIR. Preferably, combined equivalent amino acid sequences will have at least 85 percent sequence identity with the combined sequences found in SEQ. ID. NOS. 38, 40 and 42. Even more preferred are combined equivalent amino acid sequences that have at least 95 percent sequence identity with the combined amino acid sequences (SEQ. ID. NOS. 38, 40 and 42).

It is preferred that the VL CDR amino acid sequences meet both the individual equivalency and combined equivalency requirements set forth above. However, there are certain situations, especially for the shorter CDRs, where one or more of the CDRs may not meet the criteria for individual equivalence even though the criteria for combined equivalence is met. In such situations, the individual equivalency requirements are waived provided that the combined equivalency requirements are met. For example, VH CDR3 (SEQ. ID. NO. 42) is only 9 amino acids long. If three amino acids are changed, then the individual sequence identity is only 66% which is below the 75% floor for individual equivalence set forth above. However, this particular sequence is still suitable for use as part of a combined equivalent VL CDR group provided that the sequence identity of the combined CDR1, CDR2 and CDR3 sequences meet the group equivalency requirements.

The first framework region (FR1) of the VH is located at the amino end of the humanized antibody. The fourth framework region (FR4) is located towards the carboxyl end of the humanized antibody. Exemplary preferred amino acid sequences for the humanized VH FR1, FR2, FR3 and FR4 are set forth in SEQ. ID. NOS. 30, 32, 34 and 36, respectively, and these preferred sequences correspond to version 5 humanized HIRMAb (Table 3). The amino acid sequence for FR2 (SEQ. ID. NO. 32) is identical to the amino acid sequence of murine MAb 83-14 VH FR2 or the human IgG, B43 (See FIG. 4). The amino acid sequences for VH FR1 and FR4 (SEQ. ID. NOS. 30 and 36) correspond to the B43 human antibody framework regions that have amino acid sequences that differ from murine MAb 83-14 (FIG. 4). The amino acid sequences for the VH FR3 (SEQ. ID. No. 34) of the version 5 humanized HIRMAb corresponds to the VH FR3 of the murine 83-14 antibody (Table 3). It is possible to modify the preferred VH FR sequences without destroying the biological activity of the antibody. Suitable alternate or equivalent FRs include those that have at least 70 percent individual sequence identity with SEQ. ID. NOS. 30, 32, 34 or 36 and do not destroy the resulting antibodies ability to bind the HIR. Preferably, the alternate FRs will have at least 80 percent sequence identity with the preferred VH FR that is being replaced. Even more preferred are alternate FRs that have at least 90 percent sequence identity with the preferred VH FR that is being replaced.

The four VH FR amino acid sequences may also be viewed as a combined group of amino acid sequences (VH FR1, VH FR2, VH FR3 and VH FR4). The present invention also covers alternates or equivalents of the combined group of VH FR sequences. Such “combined equivalents” are those that have at least 70 percent sequence identity with the combined amino acid sequences SEQ. ID. NOS. 30, 32, 34 and 36 and which do not adversely affect the binding of the antibody to the HIR. Preferably, combined equivalent amino acid sequences will have at least 80 percent sequence identity with the combined sequences found in SEQ. ID. NOS. 30, 32, 34 and 36. Even more preferred are combined equivalent amino acid sequences that have at least 90 percent sequence identity with the combined amino acid sequences (SEQ. ID. NOS. 30, 32, 34 and 36).

It is preferred that the alternate VH FR amino acid sequences meet both the individual equivalency and combined equivalency requirements set forth above. However, there are certain situations, especially for the shorter FRs, where one or more of the FRs may not meet the criteria for individual equivalence even though the criteria for combined equivalence is met. In such situations, the individual equivalency requirements are waived provided that the combined equivalency requirements are met.

The first framework region (FR1) of the LC is located at the amino end of the VL of the humanized antibody. The fourth framework region (FR4) is located towards the carboxyl end of the VL of the humanized antibody. Exemplary preferred amino acid sequences for the humanized VL FR1, FR2, FR3 and FR4 are set forth in SEQ. ID. NOS. 37, 39, 41 and 43, respectively. The amino acid sequences for VL FR1, FR2, FR3 and FR4 (SEQ. ID. NOS. 37, 39, 41 and 43) correspond to the PEI human antibody framework regions that have amino acid sequences that differ from murine MAb 83-14 (See FIG. 4). It is possible to modify the preferred VL FR sequences without destroying the biological activity of the antibody. Suitable alternate or equivalent FRs include those that have at least 70 percent sequence identity with SEQ. ID. NOS. 37, 39, 41 and 43 and do not destroy the resulting antibodies ability to bind the HIR. Preferably, the equivalent or alternate FRs will have at least 80 percent sequence identity with the preferred VL FR that is being replaced. Even more preferred are alternate FRs that have at least 90 percent sequence identity with the preferred VL FR that is being replaced.

The four VL FR amino acid sequences may also be viewed as a combined group of amino acid sequences (VL FR1, VL FR2, VL FR3 and VL FR4). The present invention also covers alternates or equivalents of the combined group of VL FR sequences. Such “combined equivalents” are those that have at least 70 percent sequence identity with the combined amino acid sequences SEQ. ID. NOS. 37, 39, 41 and 43 and which do not adversely affect the binding of the antibody to the HIR. Preferably, combined equivalent amino acid sequences will have at least 80 percent sequence identity with the combined sequences found in SEQ. ID. NOS. 37, 39, 41 and 43. Even more preferred are combined equivalent amino acid sequences that have at least 90 percent sequence identity with the combined amino acid sequences (SEQ. ID. NOS. 37, 39, 41 and 43).

It is preferred that the alternate VL FR amino acid sequences meet both the individual equivalency and combined equivalency requirements set forth above. However, there are certain situations, especially for the shorter FRs, where one or more of the FRs may not meet the criteria for individual equivalence even though the criteria for combined equivalence is met. In such situations, the individual equivalency requirements are waived provided that the combined equivalency requirements are met.

Version 5 is a preferred humanized antibody in accordance with the present invention. The amino acid sequences for the VH and VL of Version 5 are set forth in SEQ. ID. NOS. 5 and 6, respectively. The preparation and identification of Version 5 is set forth in more detail in Example 2, Table 3 and FIG. 4. The amino acid sequences for the VH FRs of Version 5 correspond to the preferred VH FR sequences set forth above (SEQ. ID. NOS. 30, 32, 34 and 36). In addition, the amino acid sequences for the VL FRs of Version 5 correspond to the preferred VL FR sequences set forth above (SEQ. ID. NOS. 37, 39, 41, 43). The VH and VL FRs of Version 5 are a preferred example of VH and VL LC FRs that have been “humanized”. “Humanized” means that the four framework regions in either the HC or LC have been matched as closely as possible with the FRs from a human antibody (HAb) without destroying the ability of the resulting antibody to bind the HIR. The model human antibody used for the HC is the B43 antibody, and the model human antibody used for the LC is the REI antibody, and both the B43 and REI antibody sequences are well known and available in public databases. When the HC or LC FRs are humanized, it is possible that one or more of the FRs will not correspond identically with the chosen HAb template and may retain identity or similarity to the murine antibody. The degree to which murine amino acid sequences are left in the humanized FRs should be kept as low as possible in order to reduce the possibility of an immunogenic reaction in humans.

Examples of FRs that have been humanized are set forth in Example 2 and Table 3. Framework regions from human antibodies that correspond closely to the FRs of murine MAb 84-13 are chosen. The human FRs are then substituted into the MAb 84-13 in place of the murine FRs. The resulting antibody is then tested. The FRs, as a group, are only considered to be humanized if the modified antibody still binds strongly to the HIR receptor and has reduced immunogenicity in humans. If the first test is not successful, then the human FRs are modified slightly and the resulting antibody tested. Exemplary human antibodies that have HC FRs that may be used to humanize the HC FRs of MAb 84-13 include B43 human IgG (SEQ. ID. NO. 12), which is deposited in Genbank (accession number S78322), and other human IgG molecules with a VH homologous to the murine 83-14 VH may be found by searching public databases, such as the Kabat Database of immunoglobulin sequences. Exemplary human antibodies that have LC FRs that may be used to humanize the LC FRs of MAb 84-13 include human REI antibody (SEQ. ID. NO. 13), which is deposited in Genbank (accession number 1WTLB), and other human IgG molecules with a VL homologous to the murine 83-14 VL may be found by searching public databases, such as the Kabat Database of immunoglobulin sequences.

In order for the humanized antibody to function properly, the HC and LC should each include a constant region. Any number of different human antibody constant regions may be incorporated into the humanized antibody provided that they do not destroy the ability of the antibody to bind the HIR. Suitable human antibody HC constant regions include human IgG1, IgG2, IgG3, or IgG4. The preferred HC constant region is human IgG1. Suitable human antibody LC constant regions include kappa (K) or lambda. Human K LC constant regions are preferred.

The humanized antibody may be used in the same manner as any of the other antibody targeting agents (Trojan Horses) that have previously been used to deliver genes, drugs and diagnostic agents to cells by accessing the HIR. The humanized antibody is typically linked to a drug or diagnostic compound (pharmaceutical agent) and combined with a suitable pharmaceutical carrier and administered intravenously (iv). With suitable carriers, the drug/humanized antibody complex could also be administered subcutaneously, intra-muscularly, intra-nasally, intra-thecally, or orally. There are a number of ways that the humanized antibody may be linked to the pharmaceutical agent. The humanized antibody may be fused to either avidin or streptavidin and conjugated to a pharmaceutical agent that has been mono-biotinylated in accordance with known procedures that use the avidin-biotin linkage to conjugate antibody Trojan Horses and pharmaceutical agents together. Alternatively, the humanized antibody and pharmaceutical agent may be expressed as a single fusion protein using known genetic engineering procedures.

Exemplary pharmaceutical agents to which the humanized antibody may be linked include small molecules, recombinant proteins, synthetic peptides, antisense agents or nanocontainers for gene delivery. Exemplary recombinant proteins include basic fibroblast growth factor (bFGF), human α-L-iduronidase (IDUA), or other neurotrophins, such as brain derived neurotrophic factor, or other lysosomal enzymes. The use of Trojan Horses, such as the present humanized antibody, for transporting bFGF across the BBB is described in a co-pending U.S. patent application Ser. No. ______ (UC Case 2002-094-1, Attorney Docket 0180-0027) that is owned by the same assignee as the present application and which was filed on the same day as the present application).

Once the humanized antibody is linked to a pharmaceutical agent, it is administered to the patient in the same manner as other known conjugates or fusion proteins. The particular dose or treatment regimen will vary widely depending upon the pharmaceutical agent being delivered and the condition being treated. The preferred route of administration is intravenous (iv). Suitable carriers include saline or water buffered with acetate, phosphate, TRIS or a variety of other buffers, with or without low concentrations of mild detergents, such as one from the Tween series of detergents. The humanized antibody/pharmaceutical agent Trojan horse compound is preferably used to deliver neuropharmaceutical agents across the BBB. However, the humanized Trojan horse may also be used to deliver pharmaceutical agents, in general, to any organ or tissue that carries the HIR.

The following examples describe how the humanized monoclonal antibodies in accordance with the present invention were discovered and additional details regarding their fabrication and use.

### EXAMPLE 1

### Cloning of Murine 83-14 VH and VL Genes

Poly A+ RNA was isolated from the 83-14 hybridoma cell line (Soos et al, 1986), and used to produce complementary DNA (cDNA) with reverse transcriptase. The cDNA was used with polymerase chain reaction (PCR) amplification of either the 83-14 VH or 83-14 VL gene using oligodeoxynucleotide (ODN) primers that specifically amplify the VH and VL of murine antibody genes, and similar methods are well known (Li et al., 1999). The sequences of PCR ODNs suitable for PCR amplification of these gene fragments are well known (Li., 1999). The PCR products were isolated from 1% agarose gels and the expected 0.4 Kb VH and VL gene products were isolated. The VH and VL gene fragments were sequentially subcloned into a bacterial expression plasmid so as to encode a single chain Fv (ScFv) antibody. The ScFv expression plasmid was then used to transform E. Coli. Individual colonies were identified on agar plates and liquid cultures were produced in LB medium. This medium was used in immunocytochemistry of Rhesus monkey brain to identify clones producing antibody that bound avidly to the Rhesus monkey brain microvasculature or BBB. This immunocytochemistry test identified those colonies secreting the functional 83-14 ScFv. Following identification of the 83-14 VH and VL genes, the nucleotide sequence was determined in both directions using automatic DNA sequencing methods. The nucleotide sequence of the murine 83-14 VH (SEQ. ID. NO. 1) and the murine VL (SEQ. ID. NO. 2) gives the deduced amino acid sequence for the murine VH (SEQ. ID. NO. 3) and the murine VL (SEQ. ID. NO. 4). The amino acid sequence is given for all 3 CDRs and all 4 FRs of both the HC and the LC of the murine 83-14 HIRMAb. The variable region of the LC is designated VL, and the variable region of the HC is designated VH in FIG. 1.

### EXAMPLE 2

### Iterative Humanization of the 83-14 HIRMAb: Version 1 through Version 5

Humanization of the 83-14 MAb was performed by CDR/FR grafting wherein the mouse FRs in the 83-14 MAb are replaced by suitable human FR regions in the variable regions of both the LC and HC. The Kabat database was screened using the Match program. Either the murine 83-14 VH or the VL amino acid sequence was compared with human IgG VH or human K light chain VL databases. Using the minimal mismatch possible, several human IgG molecules were identified that contained FR amino sequences highly homologous to the amino acid sequences of the murine 83-14 VH and VL. The framework regions of the B43 human IgG1 heavy chain and the REI human κ light chain were finally selected for CDR/FR grafting of the murine 83-14 HIRMAb.

Sets of 6 ODN primers, of 69-94 nucleotides in length, were designed to amplify the synthetic humanized 83-14 VL and VH genes (Tables 1 and 2). The ODN primers overlapped 24 nucleotides in both the 5′- and 3′-ends, and secondary structure was analyzed with standard software. Stable secondary structure producing Tₘ of >46° C. was corrected by replacement of first, second, or third letter codons to reduce the melting point of these structures to 32-46° C. In addition, primers corresponding to both 5′ and 3′ ends were also designed, and these allowed for PCR amplification of the artificial genes. These new sequences lack any consensus N-glycosylation sites at asparagine residues.

TABLE 1
Oligodeoxynucleotides for CDR/FR grafting of VL

| | |
|---|---|
| Primer 1 FWD | |
| 5′TAGGATATCCACCATGGAGACCCCCGCCCA | (SEQ. ID. NO. 14) |
| GCTGCTGTTCCTGTTGCTGCTTTGGCTTCCAG | |
| ATACTACCGGTGACATCCAGATGACCCAG-3′ | |
| Primer 2 reverse | |
| 5′GTCCTGACTAGCCCGACAAGTAATGGTCAC | (SEQ. ID. NO. 15) |
| TCTGTCACCCACGCTGGCGCTCAGGCTGCTTG | |
| GGCTCTGGGTCATCTGGATGTCGCCGGT-3′ | |
| Primer 3 FWD | |
| 5′ATTACTTGTCGGGCTAGTCAGGACATTGGA | (SEQ. ID. NO. 16) |
| GGAAACTTATATTGGTACCAACAAAAGCCAGG | |
| TAAAGCTCCAAAGTTACTGATCTACGCC-3′ | |
| Primer 4 reverse | |
| 5′GGTGTAGTCGGTACCGCTACCACTACCACT | (SEQ. ID. NO. 17) |
| GAATCTGCTTGGCACACCAGAATCTAAACTAG | |
| ATGTGGCGTAGATCAGTAACTTTGGAGC-3′ | |
| Primer 5 FWD | |
| 5′AGTGGTAGCGGTACCGACTACACCTTCACC | (SEQ. ID. NO. 18) |
| ATCAGCAGCTTACAGCCAGAGGACATCGCCAC | |
| CTACTATTGCCTACAGTATTCTAGTTCT-3′ | |
| Primer 6 reverse | |
| 5′CCCGTCGACTTCAGCCTTTTGATTTCCACC | (SEQ. ID. NO. 19) |
| TTGGTCCCTTGTCCGAACGTCCATGGAGAACT | |
| AGAATACTGTAGGCAATA-3′ | |
| 5-PCR primer FWD | |
| 5′TAGGATATCCACCATGGAGACCCC-3′ | (SEQ. ID. NO. 20) |
| 3-PCR primer reverse | |
| 5′CCCGTCGACTTCAGCCTTTTGATT-3′ | (SEQ. ID. NO. 21) |

TABLE 2
Oligodeoxynucleotides for CDR/FR grafting of VH

| | |
|---|---|
| PRIMER 1 FWD | |
| 5′TAGGATATCCACCATGGACTGGACCTGGAG | (SEQ. ID. NO. 22) |
| GGTGTTATGCCTGCTTGCAGTGGCCCCCGGAG | |
| CCCACAGCCAAGTGCAGCTGCTCGAGTCTGGG | |
| -3′ | |
| PRIMER 2 REVERSE | |
| 5′GTTTGTGAAGGTGTAACCAGAAGCCTTGCA | (SEQ. ID. NO. 23) |
| GGAAATCTTCACTGAGGACCCAGGCCTCACCA | |
| GCTCAGCCCCAGACTCGAGCAGCTGCACTTG | |
| -3′ | |
| PRIMER 3 FWD | |
| 5′GCTTCTGGTTACACCTTCACAAACTACGAT | (SEQ. ID. NO. 24) |
| ATACACTGGGTGAAGCAGAGGCCTGGACAGGG | |
| TCTTGAGTGGATTGGATGGATTTATCCTGGA | |
| -3′ | |
| PRIMER 4 REVERSE | |
| 5′GCTGGAGGATTCGTCTGCAGTCAGAGTGGC | (SEQ. ID. NO. 25) |
| TTTGCCCTTGAATTTCTCATTGTACTTAGTAC | |
| TACCATCTCCAGGATAAATCCATCCAATCCA | |
| -3′ | |
| PRIMER 5 FWD | |
| 5′CTGACTGCAGACGAATCCTCCAGCACAGCC | (SEQ. ID. NO. 26) |
| TACATGCAACTAAGCAGCCTACGATCTGAGGA | |
| CTCTGCGGTCTATTCTTGTGCAAGAGAGTGG | |
| -3′ | |
| PRIMER 6 REVERSE | |
| 5′CATGCTAGCAGAGACGGTGACTGTGGTCCC | (SEQ. ID. NO. 27) |
| TTGTCCCCAGTAAGCCCACTCTCTTGCACAAG | |
| AATAGAC-3′ | |
| 5′-PCR PRIMER FWD | |
| 5′TAGGATATCCACCATGGACTGGACCTG-3′ | (SEQ. ID. NO. 28) |
| 3′-PRC PRIMER REV | |
| 5′CATGCTAGCAGAGACGGTGACTGTG-3′ | (SEQ. ID. NO. 29) |

The PCR was performed in a total volume of 100 μL containing 5 pmole each of 6 overlapping ODNs, nucleotides, and Taq and Taq extender DNA polymerases. Following PCR, the humanized VH and VL genes were individually ligated in a bacterial expression plasmid and E. coli was transformed. Several clones were isolated, individually sequenced, and clones containing no PCR-introduced sequence errors were subsequently produced.

The humanized VH insert was released from the bacterial expression plasmid with restriction endonucleases and ligated into eukaryotic expression vectors described previously (Coloma et al, 1992; U.S. Pat. No. 5,624,659). A similar procedure was performed for the humanized VL synthetic gene. Myeloma cells were transfected with the humanized light chain gene, and this cell line was subsequently transfected with version I of the humanized heavy chain gene (Table 3). The transfected myeloma cells were screened in a 96-well ELISA to identify clones secreting intact human IgG. After multiple attempts, no cell lines producing human IgG could be identified. Conversely, Northern blot analysis indicated the transfected cell lines produced the expected humanized 83-14 mRNA, which proved the transfection of the cell line was successful. These results indicated that version I of the humanized HIRMAb, which contains no FR amino acid substitutions, was not secreted from the cell, and suggested the humanized HC did not properly assemble with the humanized LC. Version 1 was derived from a synthetic HC gene containing FR amino acids corresponding to the 25Cl′Cl antibody (Bejcek et al, 1995). Therefore, a new HC artificial gene was prepared, which contained HC FR amino acids derived from a different human IgG sequence, that of the B43 human IgG (Bejcek et al, 1995), and this yielded version 2 of the humanized HIRMAb (Table 3). However, the version 2 humanized HIRMAb was not secreted by the transfected myeloma cell. Both versions 1 and 2 contain the same HC signal peptide (Table 3), which is derived from Rechavi et al (1983). In order to evaluate the effect of the signal peptide on IgG secretion, the signal peptide sequence was changed to that used for production of the chimeric HIRMAb (Coloma et al, 2000), and the sequence of this signal peptide is given in Table 3. Versions 2 and 3 of the humanized HIRMAb differed only with respect to the signal peptide (Table 3). However, version 3 was not secreted from the myeloma cell, indicating the signal peptide was not responsible for the lack of secretion of the humanized HIRMAb.

The above findings showed that simply grafting the murine 83-14 CDRs on to human FR regions produced a protein that could not be properly assembled and secreted. Prior work had shown that the chimeric form of the HIRMAb was appropriately processed and secreted in transfected myeloma lines (Coloma et al, 2000). This suggested that certain amino acid sequences within the FRs of the humanized HC or LC prevented the proper assembly and secretion of the humanized HIRMAb. Therefore, chimeric/humanized hybrid molecules were engineered. Version 4a contained the murine FR1 and the humanized FR2, FR3, and FR4; version 4b contained the murine FR 3, and FR4 and the humanized FR1 and FR2 (Table 3). Both versions 4a and 4b were secreted, although version 4b was more active than version 4a. These findings indicated amino acids within either FR3 or FR4 were responsible for the lack of secretion of the humanized HIRMAb. The human and murine FR4 differed by only 1 amino acid (Table 3); therefore, the sequence of FR4 was altered by site-directed mutagenesis to correspond to the human sequence, and this version was designated version 5 (Table 3). The version 5 HIRMAb corresponded to the original CDR-grated antibody sequence with substitution of the human sequence in FR3 of the VH with the original murine sequence for the FR3 in the VH. The same CDR-grafted LC, without any FR substitutions, was used in production of all versions of the humanized HIRMAb. This corresponds with other work showing no FR changes in the LC may be required (Graziano et al, 1995).

TABLE 3
Iterations of Genetic Engineering of Humanized HIRMAb Heavy Chain

| | FR1 CDR1 FR2 |
|---|---|
| Version 5 | QVQLLESGAELVRPGSSVKISCKAS GYTFTNYDIH WVKQRPGQGLEWIG |
| Version 4b | QVQLLESGAELVRPGSSVKISCKAS GYTFTNYDIH WVKQRPGQGLEWIG |
| Version 4a | QVQLQESGPELVKPGALVKISCKAS GYTFTNYDIH WVKQRPGQGLEWIG |
| Version 3 | QVQLLESGAELVRPGSSVKISCKAS GYTFTNYDIH WVKQRPGQGLEWIG |
| Version 2 | QVQLLESGAELVRPGSSVKISCKAS GYTFTNYDIH WVKQRPGQGLEWIG |
| Version 1 | QVQLLESGAELVRPGSSVKISCKAS GYTFTNYDIH WVKQRPGQGLEWIG |
| murine | QVQLQESGPELVKPGALVKISCKAS GYTFTNYDIH WVKQRPGQGLEWIG |
| human B43 | QVQLLESGAELVRPGSSVKISCKAS GYAFSSYWMN WVKQRPGQGLEWIG |
| | 1 26 36 |
| | CDR2 FR3 |
| Version 5 | WIYPGDGSTKYNEKFKG KATLTADKSSSTAYMHLSSLTSEKSAVYFCAR |
| Version 4b | WIYPGDGSTKYNEKFKG KATLTADKSSSTAYMHLSSLTSEKSAVYFCAR |
| Version 4a | WIYPGDGSTKYNEKFKG KATLTADESSSTAYMQLSSLRSEDSAVYSCAR |
| Version 3 | WIYPGDGSTKYNEKFKG KATLTADESSSTAYMQLSSLRSEDSAVYSCAR |
| Version 2 | WIYPGDGSTKYNEKFKG KATLTADESSSTAYMQLSSLRSEDSAVYSCAR |
| Version 1 | WIYPGDGSTKYNEKFKG QATLTADKSSSTAYMQLSSLTSEDSAVYSCAR |
| murine | WIYPGDGSTKYNEKFKG KATLTADKSSSTAYMHLSSLTSEKSAVYFCAR |
| human B43 | QIWPGDGDTNYNGKFKG KATLTADESSSTAYMQLSSLRSEDSAVYSCAR |
| | 50 67 |

| | | |
|---|---|---|
| | CDR3 FR4 | |
| Version 5 | -----------EWAY WGQGTTVTVSA | (SEQ. ID. NO. 5) |
| Version 4b | -----------EWAY WGQGTLVTVSA | (SEQ. ID. NO. 11) |
| Version 4a | -----------EWAY WGQGTTVTVSA | (SEQ. ID. NO. 10) |
| Version 3 | -----------EWAY WGQGTTVTVSA | (SEQ. ID. NO. 9) |
| Version 2 | -----------EWAY WGQGTTVTVSA | (SEQ. ID. NO. 8) |
| Version 1 | -----------EWAY WGQGTTVTVSA | (SEQ. ID. NO. 7) |
| murine | -----------EWAY WGQGTLVTVSA | (SEQ. ID. NO. 3) |
| human B43 | RETTTVGRYYYAMDY WGQGTTVT--- | (SEQ. ID. NO. 12) |
| | 99 99 103 113 | |

Version 1 was designed using the FRs of the human 25ClCl IgG heavy chain (HC) variable region (VH). Version 1 did not produce secreted hIgG from the transfected myeloma cells despite high abundance of the HC mRNA determined by Northern blot analysis.

Version 2 was re-designed using the FRs of the human B43 IgG HC variable region. The peptide signal #1 (MDWTWRVLCLLAVAPGAHS) (SEQ. ID. NO. 49) in versions 1 and 2 was replaced by signal peptide #2 (MGWSWVMLFLLSVTAGKGL) (SEQ. ID. NO. 50) in version 3. The FRs and CDRs in version 2 and 3 are identical. The signal peptide #2 was used for versions 4a, 4b and 5.

Verson 4a has human FRs 2, 3 and 4 and murine FR1.

Version 4b has human FRs 1 and 2, and murine FRs 3 and 4.

Version 5 was produced using the human FRs 1, 2 and 4 and the murine FR3.

Versions 4a, 4b and 5 produced secreted hIgG, whereas version 1, 2, and 3 did not secrete IgG. Among versions 4a, 4b, and 5, version 5 contains fewer murine framework amino acid substitutions and is preferred.

The version 5 form of the protein was secreted intact from the transfected myeloma lines. The secreted version 5 humanized HIRMAb was purified by protein A affinity chromatography and the affinity of this antibody for the HIR was tested with an immunoradiometric assay (IRMA), which used [¹²⁵I]-labeled murine 83-14 MAb as the ligand as described previously (Coloma et al, 2000). These results showed that the affinity of the antibody for the HIR was retained. In the IRMA, the antigen was the extracellular domain of the HIR, which was produced from transfected CHO cells and purified by lectin affinity chromatography of CHO cell conditioned medium. The dissociation constant (K_{D}) of the murine and Version 5 humanized 83-14 HIRMAb was 2.7±0.4 nM and 3.7±0.4 nM, respectively. These results show that the 83-14 HIRMAb has been successfully humanized using methods that (a) obtain the FR regions of the HC and of the LC from different human immunoglobulin molecules, and (b) do not require the use of molecular modeling of the antibody structure, as taught in U.S. Pat. No. 5,585,089. Similar to other applications (Graziano et al., 1995), no FR amino acid changes in the LC of the antibody were required.

### EXAMPLE 3

### Binding of the Humanized HIRMAb to the Human BBB

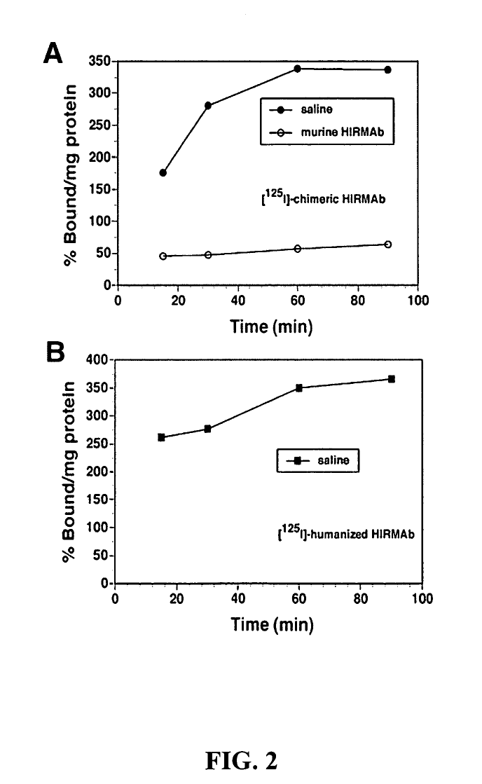

Prior work has reported that the radiolabelled murine HIRMAb avidly binds to human brain capillaries with percent binding approximately 400% per mg protein at 60-120 minutes of incubation (Pardridge et al., 1995). Similar findings were recorded with radiolabelled Version 5 humanized HIRMAb in this example. When human brain capillaries were incubated in a radioreceptor assay with [¹²⁵I] Version 5 humanized HIRMAb, the percent binding approximated 400% per mg protein by 60 minutes of incubation at room temperature, and approximated the binding to the human brain capillary of the [¹²⁵I-chimeric HIRMAb (see FIGS. 2A and 2B). In contrast, the binding of a nonspecific IgG to human brain capillaries is less than 5% per mg protein during a comparable incubation period Pardridge et al., 1995). This example shows that the Version 5 humanized HIRMAb was avidly bound and endocytosed by the human brain capillary, which forms the BBB in vivo.

### EXAMPLE 4

### Transport of Humanized HIRMAb Across the Primate BBB In Vivo

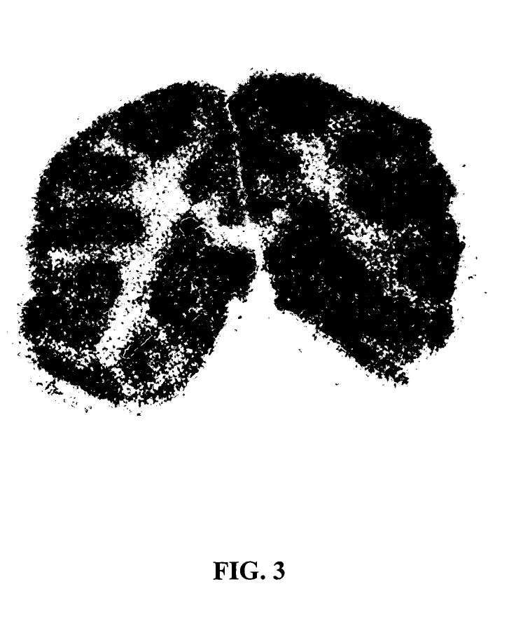

The humanized Version 5 HIRMAb was radiolabelled with 125-Iodine and injected intravenously into the adult Rhesus monkey. The animal was sacrificed 2 hours later and the brain was removed and frozen. Cryostat sections (20 micron) were cut and applied to X-ray film. Scanning of the film yielded an image of the primate brain uptake of the humanized HIRMAb (FIG. 3). The white matter and gray matter tracts of the primate brain are clearly delineated, with a greater uptake in the gray matter as compared with the white matter. The higher uptake of the human HIRMAb in the gray matter, as compared with the white matter, is consistent with the 3-fold higher vascular density in gray matter, and 3-fold higher nonspecific IgG is injected into Rhesus monkeys there is no brain uptake of the antibody (Pardridge et al., 1995). These film autoradiography studies show that the humanized HIRMAb is able to carry a drug (iodine) across the primate BBB in vivo. Based on the high binding of the humanized HIRMAb to the human BBB (FIG. 2), similar findings of high brain uptake in vivo would be recorded in humans.

### EXAMPLE 5

### Affinity Maturation of the Antibody by CDR or FR Amino Acid Substitution

The amino acid sequences of the VH of the HC and of the VL of the LC are given in FIG. 4 for the Version 5 humanized HIRMAb, the murine 83-14 HIRMAb, and either the B43 HC or the REI LC antibodies. Given the CDR amino sequences in FIG. 4, those skilled in the art of antibody engineering (Schier et al., 1996) may make certain amino acid substitutions in the 83-14 HC or LC CDR sequences in a process called “affinity maturation” or molecular evolution. This may be performed either randomly or guided by x-ray diffraction models of immunoglobulin structure, similar to single amino acid changes made in the FR regions of either the HC or the LC of an antibody (U.S. Pat. No. 5,585,089). Similarly, given the FR amino acid sequences in FIG. 4, those skilled in the art can make certain amino acid substitutions in the HC or LC FR regions to further optimize the affinity of the HIRMAb for the target HIR antigen. The substitutions should be made keeping in mind the sequence identity limitations set forth previously for both the FR and CDR regions. These changes may lead to either increased binding or increased endocytosis or both.

### EXAMPLE 6

### Humanized HIRMAb/α-L-iduronidase Fusion Protein

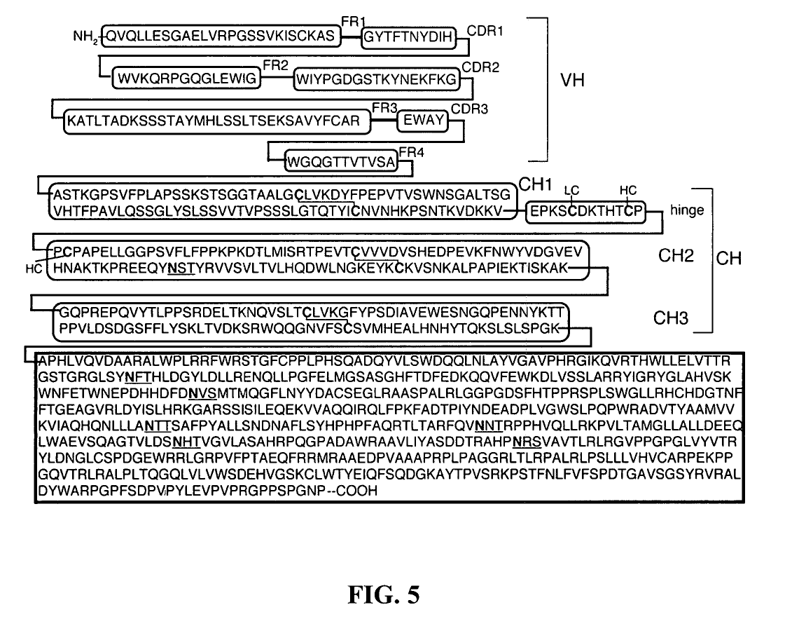

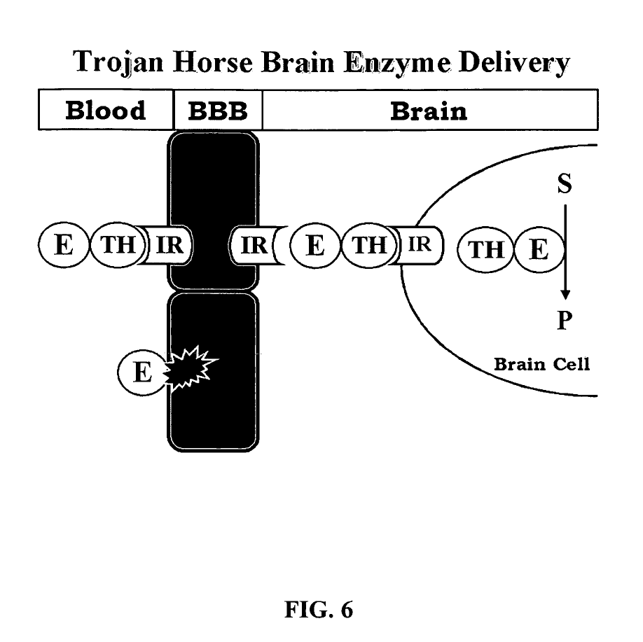

α-L-iduronidase (IDUA) is the enzyme missing in patients with Hurler syndrome or type I mucopolysaccharidosis (MPS), which adversely affects the brain. The brain pathology ultimately results in early death for children carrying this genetic disease. IDUA enzyme replacement therapy (ERT) for patients with MPS type I is not effective for the brain disease, because the enzyme does not cross the BBB. This is a serious problem and means the children with this disease will die early even though they are on ERT. The enzyme could be delivered across the human BBB following peripheral administration providing the enzyme is attached to a molecular Trojan horse such as the humanized HIRMAb. The IDUA may be attached to the humanized HIRMAb with avidin-biotin technology. In this approach, the IDUA enzyme is mono-biotinylated in parallel with the production of a fusion protein of the humanized HIRMAb and avidin. In addition, the IDUA could be attached to the humanized HIRMAb not with avidin-biotin technology, but with genetic engineering that avoids the need for biotinylation or the use of foreign proteins such as avidin. In this approach, the gene encoding for IDUA is fused to the region of the humanized HIRMAb heavy chain or light chain gene corresponding to the amino or carboxyl terminus of the HIRMAb heavy or light chain protein. Following construction of the fusion gene and insertion into an appropriate prokaryotic or eukaryotic expression vector, the HIRMAb/IDUA fusion protein is mass produced for purification and manufacturing. The amino acid sequence and general structure of a typical MAb/IDUA fusion protein is shown in FIG. 5 (SEQ. ID. NO. 48). In this construct, the enzyme is fused to the carboxy terminus of the heavy chain (HC) of the humanized HIRMAb. The amino acid sequence for the IDUA shown in FIG. 5 is that of the mature, processed enzyme. Alternatively, the enzyme could be fused to the amino terminus of the HIRMAb HC or the amino or carboxyl termini of the humanized HIRMAb light chain (LC). In addition, one or more amino acids within the IDUA sequence could be modified with retention of the biological activity of the enzyme. Fusion proteins of lysosomal enzymes and antibodies have been prepared and these fusion proteins retain biological activity (Haisma et al, 1998). The fusion gene encoding the fusion protein can be inserted in one of several commercially available permanent expression vectors, such as pCEP4, and cell lines can be permanently transfected and selected with hygromycin or other selection agents. The conditioned medium may be concentrated for purification of the recombinant humanized HIRMAb/IDUA fusion protein.

### EXAMPLE 7

### Role of Light Chain (LC) in Binding of HIRMAb to the Human Insulin Receptor

Myeloma cells (NSO) were transfected with a plasmid encoding the either the humanized HIRMAb light chain or “surrogate light chain”, which was an anti-dansyl MAb light chain (Shin and Morrison, 1990). The anti-dansyl light chain is derived from the anti-dansyl IgG, where dansyl is a common hapten used in antibody generation. Both the myeloma line transfected with the humanized HIRMAb light chain, and the myleoma line transfected with the surrogate light chain were subsequently transfected with a plasmid encoding the heavy chain of the chimeric HIRMAb. One cell line secreted an IgG comprised of the anti-HIRMAb chimeric heavy chain and the anti-HIRMAb humanized light chain, and this IgG is designated chimeric HIRMAb heavy chain/humanized HIRMAb light chain IgG. The other cell line secreted an IgG comprised of a chimeric HIRMAb heavy chain and the anti-dansyl light chain, and this IgG is designated chimeric HIRMAb HC/dansyl LC IgG. Both cells lines secreted IgG processed with either the humanized HIRMAb light chain or the anti-dansyl light chain, as determined with a human IgG ELISA on the myeloma supernatants. These data indicated the chimeric HIRMAb heavy chain could be processed and secreted by myeloma cells producing a non-specific or surrogate light chain. The reactivity of these chimeric antibodies with the soluble extracellular domain (ECD) of the HIR was determined by ELISA. The HIR ECD was purified by lectin affinity chromatography of the conditioned medium of CHO cells transfected with the HIR ECD as described previously (Coloma et al, 2000). In the HIR ECD ELISA, the murine 83-14 HIRMAb was used as a positive control and mouse IgG2a was used as a negative control. The negative control produced negligible ELISA signals; the standard curve with the murine 83-14 MAb gave a linear increase in absorbance that reached saturation at 1 μg/ml murine 83-14 MAb. The immune reaction in the ELISA was quantified with a spectrophotometer and maximum absorbance at 405 nm (A405) in this assay was 0.9. All isolated myeloma clones secreting the chimeric HIRMAb heavy chain/humanized HIRMAb light chain IgG were positive in the HIR ECD ELISA with immuno-reactive levels that maximized the standard curve. In addition, the myeloma clones secreting the chimeric HIRMAb HC/dansyl LC IgG also produced positive signals in the HIR ECD ELISA, and the A405 levels were approximately 50% of the A405 levels obtained with the chimeric HIRMAb heavy chain/humanized HIRMAb light chain IgG. These findings indicate the light chain plays a minor role in binding of the HIRMAb to its target antigen, which is the extracellular domain of the human insulin receptor. This interpretation is supported by the finding that no FR substitutions in the humanized LC were required to enable active binding of the humanized HIRMAb to the HIR ECD (see Example 2). These findings show that large variations in the amino acid sequence of the HIRMAb light chain (50% and more) can be made with minimal loss of binding of the intact humanized HIRMAb to the target HIR antigen. Accordingly, a wide variety of LC's may be used to prepare humanized antibodies in accordance with the present invention provided that they are compatible with the HC. The LC is considered to be “compatible” with the HC if the LC can be combined with the HC and not destroy the ability of the resulting antibody to bind to the HIR. In addition, the LC must be human or sufficiently humanized so that any immunogenic reaction in humans is minimized. Routine experimentation can be used to determine whether a selected human or humanized LC sequence is compatible with the HC.

Lysosomal storage disorders are treated with recombinant enzyme replacement therapy (ERT). The majority of lysosomal storage disorders affect the brain (Cheng and Smith, 2003). A major limitation in the ERT of lysosomal storage disorders is the lack of transport of the therapeutic enzyme across the brain capillary wall, which forms the blood-brain barrier (BBB). The involvement of the central nervous system is generally severe in lysosomal storage disorders (Cheng and Smith, 2003), and it is important to develop BBB drug delivery strategies for therapeutic enzymes. Recombinant proteins as large as 40,000 Daltons have been delivered across the BBB in vivo with molecular Trojan horses that access endogenous BBB receptor-mediated transport systems (Pardridge, 2001). A peptidomimetic monoclonal antibody (MAb) to the BBB transferrin receptor (TfR) mediated the delivery of several peptides and recombinant proteins across the BBB with in vivo CNS pharmacological effects following intravenous administration (Pardridge, 2001). The recombinant protein is attached to the TfRMAb via avidin-biotin technology. In this approach, the non-transportable protein drug is mono-biotinylated in parallel with the production of a TfRMAb-streptavidin (SA) conjugate. Owing to the very high affinity of SA binding of biotin, there is instantaneous formation of the protein-TfRMAb conjugate following mixing of the mono-biotinylated drug and the TfRMAb-SA (Pardridge, 2001).

As mentioned above, the HIRMAb may be used as a BBB targeting agent to deliver lysosomal enzymes, such as IDUA, across the BBB. Lysosomal enzymes have a molecular weight of 50-100 kDa (see GenBank accession numbers in Table 4 for detailed molecular weights). As another aspect of the present invention, the HIRMAb may be used to deliver lysosomal enzymes of the type listed in Table 4 and other large enzymes across the BBB. The term “large enzyme” or “high MW enzyme” as used herein means enzymes having monomer molecular weights of 40,000 Daltons to 150,000 Daltons or higher and preferably 40,000 Daltons to 150,000 Daltons. In addition, other known BBB targeting agents (also referred to herein as “Trojan horses”), such as endogenous peptides or modified proteins, including endogenous peptides, such as transferrin, insulin, leptin, insulin-like growth factors (IGFs), or cationic peptides, or peptidomimetic monoclonal antibodies to the BBB transferrin receptor, insulin receptor, IGF receptor, or leptin receptor may be used to deliver enzymes of the type and size-range mentioned above across the BBB.

Bacterial β-galactosidase (GLB) is used herein as an exemplary lysosomal enzyme to demonstrate the above-described aspect of the present invention regarding delivery of large enzymes (MW of 40,000 or more) using the HIRMAb or another suitable Trojan horse. The human β-galactosidase is a lysosomal enzyme, and mutations in the gene encoding for β-galactosidase can lead to 2 different forms of lyosomal storage disorder, MPS-IVB or Morquio Syndrome, or the GM1-gangliosidosis (Table 4). The β-galactosidase enzyme is delivered to the brain of mice with the rat 8D3 MAb to the mouse TfR, which enters brain via the BBB TfR (Lee et al, 2000).

GLB is a large enzyme with a MW of 116,000 Daltons in the monomeric configuration. Similar to GUSB, this enzyme exists as a homo-tetramer with a MW>400,000 Daltons (Juers et al, 2000). Both GUSB and GLB are enzymatically active as a monomer or dimer (Datla et al, 1991). Using amino acid alignment software, it can be shown that bacterial β-galactosidase (GenBank accession number P00722) has significant amino acid homology with human β-galactosidase (GenBank accession number P16278). The model BBB molecular Trojan horse used is a rat MAb to the mouse TfR, designated TfRMAb. The β-galactosidase was joined to the TfRMAb with avidin-biotin technology.

In this approach, the β-galactosidase was mono-biotinylated, and formulated in 1 vial. In parallel, recombinant streptavidin (SA) was joined to the TfRMAb via a stable thiol-ether linkage, and the TfRMAb/SA conjugate was formulated in a second vial. Prior to intravenous administration, the 2 vials were mixed. Owing to the very high affinity of SA binding of biotin (Green, 1975), the mono-biotinylated enzyme is rapidly conjugated to the TfRMAb, as taught in U.S. Pat. No. 6,287,792, to form the GLB/TfRMAb chimeric peptide. Following intravenous injection of the GLB alone and without the Trojan horse, there was no increase in brain β-galactosidase enzyme activity. However, following intravenous injection of the GLB/TfRMAb chimeric peptide, a 10-fold increase in brain β-galactosidase enzyme activity was observed. In addition, conjugation of the β-galactosidase to the molecular Trojan horse resulted in a marked increase in β-galactosidase enzyme activity in peripheral tissues, such as liver, spleen, and kidney. Therefore, attachment of a model lysosomal enzyme to a model BBB molecular Trojan horse solves a major medical problem—delivery of therapeutic enzymes across an intact BBB. The Trojan horse technology has the added benefit of also markedly increasing enzyme uptake into many non-brain organs.

TABLE 4
Inborn Errors of Metabolism: Candidates for CNS Enzyme Replacement Therapy

| Group | Disease | Enzyme and Gene Name | Genbank |
|---|---|---|---|
| MPS | MPS-I (Hurler) | α-L-iduronidase (IDUA) | NM_000203 |
| | MPS-II (Hunter) | iduronate-2-sulphatase (IDS) | NM_000202 |
| | MPS-III (Sanfillipo) | IIIA: N-sulfatase (SGSH) | NM_000199 |
| | | IIIB: α-N-acetylglucosaminidase | NM_000263 |
| | | (NAGLU) | |
| | MPS-IV (Morquio) | A: N-acetyl-galactosamine- | NM_000512 |
| | | 6-sulfatase (GALNS) | |
| | | B: β-galactosidase (GLB1) | NM_000404 |
| | MPS-VI | arylsulphatase B (ARSB) | NM_000046 |
| | (Maroteaux-Lamy) | | |
| | MPS-VII (Sly) | β-glucuronidase (GUSB) | NM_000181 |
| GSD | GSD-II (Pompe) | acid α-glucosidase (GAA) | NM_000152 |
| SL | Gaucher Type 2 or 3 | glucocerebrosidase | M16328 |
| | Fabry | α-galactosidase A (GLA) | NM_000169 |
| | Tay Sachs | hexosaminidase A (HEXA) | NM_000520 |
| | Niemann-Pick type A | acid sphingomyelinase (SMPD1) | NM_000543 |
| | Krabbe | β-galactocerebrosidase (GALC) | NM_000153 |
| | GM1-gangliosidosis | β-galactosidase (GLB1) | NM_000404 |
| | MLD | arylsulfatase A (ARSA) | NM_000487 |
| | Farber | acid ceramidase | U70063 |
| LD | Canavan | aspartoacylase (ASPA) | NM_000049 |
| NCL | Type 1 | palmitoyl-protein thioesterase 1 (PPT1) | NM_000310 |
| | Type 2 | tripeptidyl amino peptidase 1 (TPP1) | NM_000391 |

MPS: mucopolysaccharidosis;
GSD: glycogen storage disease;
MLD, metachromatic leukodystrophy;
NCL: neuronal ceroid lipofuscinoses;
SL: sphingolipidoses;
LD: leukodystrophy

Examples of practice are as follows:

### EXAMPLE 8

### Attachment of Enzyme to Trojan Horse with Preservation of Enzyme Activity

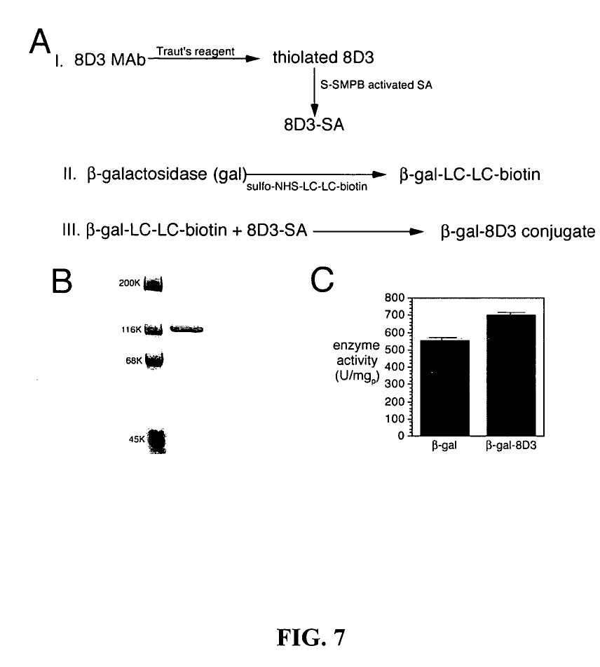

Following attachment of the enzyme to the Trojan horse, it is essential that the enzyme activity be preserved. In this prototype example, the model enzyme, β-galactosidase, was conjugated to the model Trojan horse, the rat 8D3 MAb to the mouse Tfr, via avidin-biotin technology, as outlined in FIG. 7A.

Formation of the TfRMAb/SA conjugate. The rat hybridoma line secreting the 8D3 MAb to the mouse TfR was cultured on a feeder layer of mouse thymocytes and peritoneal cells in Dulbecco modified Eagle medium with 10% fetal bovine serum (Lee et al, 2000). The 8D3 MAb was purified by protein G affinity chromatography. A 1:1 conjugate of the 8D3 MAb and streptavidin (SA) was prepared by stable thiol-ether linkage using 8D3 thiolated with Traut's reagent at a 40:1 molar ratio of Traut's reagent. The SA was activated with sulfosuccinimidyl-4-(p-malimidophenyl)butyrate (S-SMPB) at a 24:1 molar ratio, and the 8D3/SA conjugate was purified with a 2.5×95 cm column of Sephacryl S-300HR in PBST (0.01 M Na₂HPO₄, 0.15 M NaCl, pH=7.4, 0.05% Tween-20). The elution of the 8D3/SA conjugate and unconjugated SA were monitored by adding a trace amount of [³H]-biotin to the mixture prior to addition to the column. The fractions containing the 8D3/SA conjugate (FIG. 7A, reaction I) were pooled and stored at −20° C.

Mono-biotinylation of β-galactosidase and biotin quantitation. Bacterial β-galactosidase was homogeneous on sodium dodecyl sulfate polyacrylamide gel electrophoresis (SDS-PAGE), and migrated with a molecular weight (MW) of 116,000 Da (FIG. 7B). The β-galactosidase was dissolved in 0.05 M NaHCO₃/8.5 and the protein concentration was determined with the bicinchoninic acid (BCA) assay. The sulfo-NHS-LC-LC-biotin, 45 nmol/μl, was prepared in 0.05 M NaHCO₃/8.5, and 19 μl of sulfo-NHS-LC-LC-biotin solution (855 nmol) was added to 5 mg (43 nmol) of α-galactosidase, which was 20:1 molar ratio of biotin: β-galactosidase; LC=long chain, and NHS=N-hydroxysuccinimide. The mixture was capped and rocked end over end for 60 min at room temperature. The sample was applied to a 0.7×15 cm Sephadex G-25 column, eluted with 10 ml of 0.01 M PBS/7.4 at 0.5 ml/min, and 0.5 ml fractions were collected. The 3 fractions comprising the first A280 peak were pooled, the protein concentration was determined, and the biotin-LC-LC-β-galactosidase (FIG. 7A, reaction II) was stored at −20 C. The enzymatic activity of β-galactosidase or biotinylated beta-gal (biotin-LC-LC-β-galactosidase) was measured with either a spectrophotometric method or a luminescence assay system.

The molar ratio of sulfo-NHS-LC-LC-biotin to β-galactosidase was determined to yield 1-1.5 biotin moieties per enzyme molecule. The degree of biotinylation was quantified with measurements of the binding 2-(4′-hydroxyazobenzene)benzoic acid (HABA) to avidin by absorbance at 500 nm with an extinction coefficient of 34 mM⁻¹. The displacement of HABA from avidin is proportional to the biotin content in the biotin-LC-LC-β-galactosidase.

The β-galactosidase/8D3 conjugate, also designated β-gal-8D3 (FIG. 7, reaction III), was formed by mixing a 1:1 molar ratio of biotin-LC-LC-β-galactosidase and the 8D3/SA conjugate at 15 min at room temperature. There was no loss in β-galactosidase enzyme activity following mono-biotinylation and attachment to the 8D3/SA conjugate (FIG. 7C).

### EXAMPLE 9

### Trojan Horse Delivery of Enzyme to Brain with Intravenous Administration

Adult female BALB/c mice weighing 20-25 g were anesthetized with 100 mg/kg ketamine and 10 mg/kg xylazine intra-peritoneal. The mice were injected via the jugular vein with either unconjugated β-galactosidase (β-gal) or the β-gal-8D3 conjugate. In the high dose treatment, mice were administered either (a) 150 μg/mouse of unconjugated β-galactosidase, or (b) 150 μg/mouse of biotinylated α-galactosidase conjugated to 300 μg/mouse of 8D3/SA. In the low dose treatment, mice were administered either (a) 15 μg/mouse of unconjugated β-galactosidase, or (b) 15 μg/mouse of biotinylated β-galactosidase conjugated to 30 μg/mouse of 8D3/SA. The mice were sacrificed at either 1 or 4 hours after intravenous (IV) injection. The brain, liver, spleen, heart and kidney were removed, weighed and frozen on dry ice. The blood from each mouse was collected, heparinized and stored at −20 C. Organs and blood were also removed from un-injected mice to determine the activity of endogenous β-galactosidase at pH=7.4.

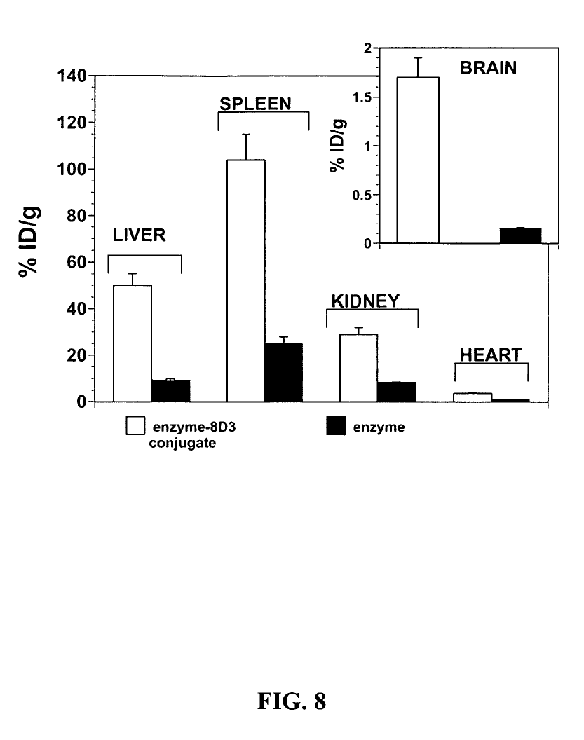

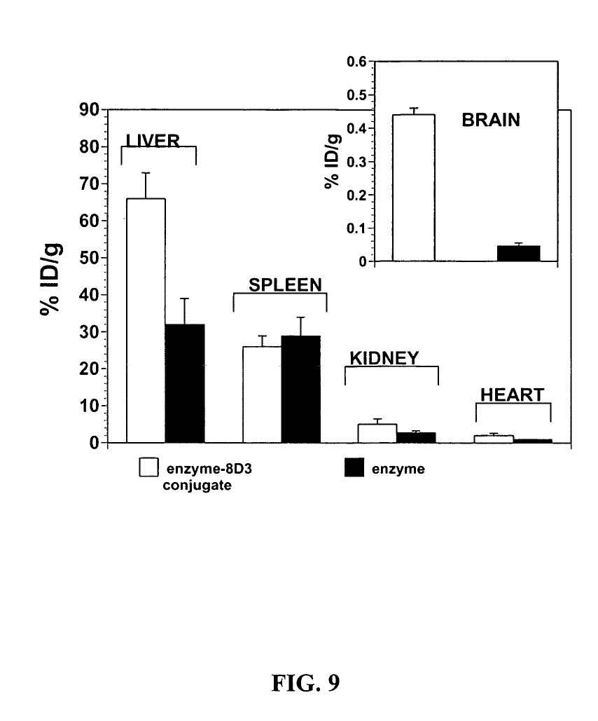

Following the IV administration of a low dose (15 ug/mouse) of the unconjugated β-galactosidase, the enzyme was rapidly cleared from blood by liver, spleen, and kidney (FIG. 8). The enzyme was cleared by liver and spleen after the IV administration of the high dose (150 ug/mouse) of the unconjugated β-galactosidase (FIG. 9). The high dose caused minimal saturation of the uptake of the unconjugated enzyme by liver and spleen. The 60 min enzyme activity in liver was 1,144±190 and 41,086±8,497 mU/g after the IV injection of the low dose and high dose, respectively. The 60 min enzyme activity in spleen was 3,038±384 and 32,686±5,777 mU/g after the IV injection of the low dose and high dose, respectively. The brain uptake of the unconjugated enzyme was minimal at both the low dose (FIG. 8, inset) and the high dose of enzyme (FIG. 9, inset). The 60 min enzyme activity in brain was 121±3 and 116±26 mU/g after the IV injection of the low dose and high dose, respectively, and both values approximated the endogenous enzyme activity in the un-injected mouse brain, 85±3 mU/g.

Conjugation of the enzyme to the TfRMAb accelerated uptake in peripheral tissues with the highest uptake by liver and spleen at the low dose of enzyme (FIG. 8). At the low dose, the brain uptake of β-galactosidase was increased 10-fold following conjugation to the TfRMAb (FIG. 8, inset). At the high dose, the uptake of the enzyme-TfRMAb conjugate by liver and spleen showed saturation (FIG. 9), whereas the brain uptake was still increased 10-fold following conjugation to the TfRMAb (FIG. 9, inset).

β-galactosidase enzyme activity measurements. A spectrophotometric assay for β-galactosidase enzyme activity was not used owing to interference in the absorbance readings by endogenous tissue pigments. Enzyme activity was measured with standard, luminescence assay system. The tissue was extracted with lysis buffer at a ratio of 2 ml buffer to 0.5 g tissue, followed by homogenization with a Polytron PT3000. The homogenate was centrifuged for 10 min at 12,000 g, and the supernatant was used to measure β-galactosidase activity with the assay solution at pH=7.6. The mixture was incubated in the dark at room temperature for 1 hour. The relative light units (RLU) were measured with a luminometer, and the RLU was converted to milliunits (mU) of enzyme activity based on a β-galactosidase standard curve. The protein content in the organ extract was measured with the BCA reagent. Organ enzyme activity was measured as: (a) mU/mg protein, (b) mU/gram organ weight, or (c) % injected dose (ID)/g organ weight. The ID was computed from the known specific activity (mU/μg) of the unconjugated β-galactosidase or the β-gal-8D3 conjugate. The endogenous β-galactosidase enzyme activity in un-injected mice was also measured in each organ.

### EXAMPLE 10

### Histochemistry of Brain Following Trojan Horse-Mediated Enzyme Delivery

The measurements of enzyme activity reported in FIGS. 8 and 9 were recorded with a highly sensitive luminescence assay. The use of a histochemical assay would have the advantage of providing a morphologic representation of enzyme delivery to brain. However, a histochemical assay is a colorimetric assay of low sensitivity. Because of the low sensitivity of the histochemical assay, mice were injected with maximal doses of either of unconjugated β-galactosidase (300 μg/mouse) or the β-gal-8D3 conjugate (300 μg/mouse of biotin-LC-LC-β-galactosidase mixed with 600 μg/mouse of 8D3/SA conjugate) via the jugular vein. At 60 min after IV injection, the brain plasma volume was cleared with a 4 min infusion of 4 mL cold PBS into the ascending aorta at a rate of 1 mL/min, followed by a 20 min perfusion of 20 ml of fixative (2% paraformaldehyde in 0.01 M PBS/7.4 with 0.5% glutaraldehyde and 2 mM MgCl₂) at a rate of 1 ml/min.

The brain was removed and divided into 4 coronal slabs, and the slabs were immersion-fixed in the same fixative at 4° C. for 4 hours. The tissue was washed briefly in 0.1 M phosphate-buffered water (PBW)/7.4 and then placed in 30% sucrose/0.1 M PBS/7.4 for 24 hours at 4° C. The brain slab was frozen in Tissue-Tek O.C.T. compound and stored at −70° C. until sectioning. Frozen sections of 40 μm were prepared on a freezing microtome at −18 C, and β-galactosidase histochemistry was performed. The frozen section was fixed with 2% formaldehyde and 0.2% glutaraldehyde in 0.01 M PBS/7.4 for 5 min. After washing in PBS, the section was incubated in X-gal staining solution (4 mM potassium ferricyanide, 4 mM potassium ferrocyanide, 2 mM MgCl₂, 0.02% IGEPAL CA-630, 0.01% sodium deoxycholate and 1 mg/ml X-gal, pH 7.4) at 37° C. overnight, where X-gal=5-bromo-4-chloro-3-indoyl-β-D-galactoside. The pH of the incubation was maintained at 7.4 throughout the incubation. After staining with X-gal, the section was briefly washed in distilled water, mounted without counter-staining, and photographed.

A dot-blot assay was developed to determine the minimal β-galactosidase enzyme activity that could be detected with a colorimetric histochemical assay. Enzyme (100 uL) was spotted with a Biorad dot blot apparatus in a 3 mm circle to nitrocellulose filter paper in the following amounts: 68, 6.8, 0.68, 0.068, and 0.0068 mU with or without fixation of the blotted filter paper in 0.2% glutaraldehyde in 0.1 M Na₂HPO₄/7.4/2 mM MgCl₂ for 2 min. Enzyme activity in the filter paper was measured with the standard colorimetric technique. The amount of enzyme that was barely detected by eye was >2 mU with fixation and >1 mU without fixation. A 40 micron section of mouse brain weighs approximately 1 mg. Therefore, it would be necessary to achieve a β-galactosidase enzyme activity >2,000 mU/g brain in order to visualize the enzyme in brain parenchyma with a colorimetric technique such as histochemistry.

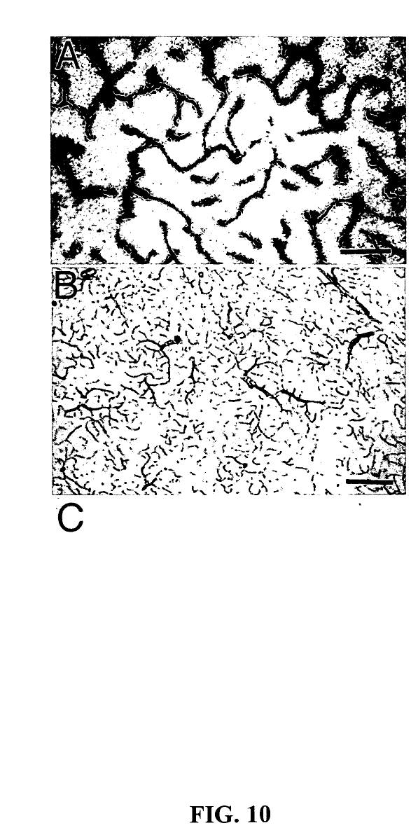

The brain uptake of the unconjugated β-galactosidase or the β-galactosidase-TfRMAb conjugate was measured with histochemistry after treatment with maximal doses. At 60 min after an IV injection of the unconugated enzyme, there is no measurable enzyme activity in brain in either the parenchymal or capillary compartment (FIG. 10C). At 60 min after an IV injection of the high dose of the β-galactosidase-TfRMAb conjugate, the enzyme product is detected by histochemistry in the capillary compartment throughout the entire brain, including cerebellum (data not shown) and a representative low magnification view is shown in FIG. 10B. High magnification microscopy (FIG. 10A) shows the enzyme within the microvascular endothelium; this enzyme activity is localized to the intra-endothelial compartment, and not the plasma compartment, because the brain was saline cleared prior to perfusion fixation for histochemistry. The brain vasculature was effectively cleared of enzyme as shown by the absence of vascular enzyme product following injection of the un-conjugated enzyme (FIG. 10C). Histochemical product in brain parenchyma was not visually detectable, because the brain β-galactosidase enzyme activity, about 500-700 mU/g, was less than the threshold for colorimetric detection, 2000 mU/g.

### EXAMPLE 11

### Confirmation of Enzyme Delivery to Brain with the Capillary Depletion Method

The delivery of enzyme into brain parenchyma with the Trojan horse, and beyond the BBB, was demonstrated with the capillary depletion technique and a luminescence-based assay of brain β-galactosidase enzyme activity. Mice were anesthetized and injected with the β-gal-8D3 conjugate (150 μg/mouse of biotin-LC-LC-β-galactosidase mixed with 300 μg/mouse of 8D3/SA conjugate) via the jugular vein. At 60 min after IV injection, residual enzyme in the brain plasma compartment was eliminated with a 4 min infusion of 4 mL cold PBS into the ascending aorta at a rate of 1 mL/min. The brain was removed, weighed and homogenized in a cold physiological buffer (10 mM HEPES, 141 mM NaCl, 4 mM KCl, 2.8 mM CaCl₂, 1 mM MgSO₄, 1 mM NaH₂O₄, and 10 mM D-glucose, pH 7.4) with a glass tissue grinder, followed by the addition of cold dextran to a final concentration of 40%. After removal of an aliquot of the homogenate, the remainder was centrifuged at 3,200 g for 10 min at 4° C. and the supernatant was carefully separated from the capillary pellet with the capillary depletion technique described previously (Triguero et al, 1990).

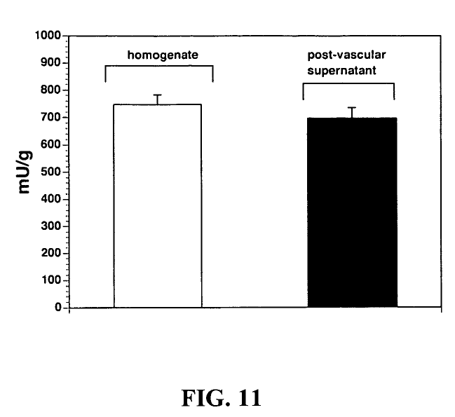

The homogenate, post-vascular supernatant, and the capillary pellet were solubilized in buffer. The β-galactosidase enzymatic activity was measured with the luminescence assay system and reported as mU/gram brain for the different fractions. More than 90% of the brain β-galactosidase enzyme activity was localized to the post-vascular supernatant compartment at 60 minutes following intravenous administration of the high dose of the β-galactosidase-TfRMAb conjugate (FIG. 11).

The β-galactosidase used in these Examples is 116 kDa (FIG. 7B) and is rapidly taken up by liver and spleen following IV injection, even in the absence of attachment to the molecular Trojan horse (FIGS. 8-9). In contrast, the brain uptake of unconjugated βgalactosidase is nil, as shown by the absence of any change in brain enzyme activity following injection of the low and high enzyme doses. Therefore, this model enzyme mimics the clinical results with conventional ERT, i.e, enzyme is taken up by certain peripheral tissues such as liver or spleen, but is not taken up by brain. The failure of the enzyme to enter the brain is a very serious problem in the treatment of lysosomal storage disorders that affect the central nervous system (CNS). Without treatment of the brain, the patients are ultimately destined to progressive neurodegeneration and early death.

These Examples show that if a 116,000 Dalton enzyme, β-galactosidase, is attached to a BBB molecular Trojan horse, there is a 10-fold increase in brain enzyme activity, at either the low or high dose treatments (FIGS. 8 and 9). When brain enzyme activity is expressed per gram brain tissue, the peak β-galactosidase enzyme activity in brain was 484±62 mU/g brain at 60 minutes following the IV injection of the high dose of the β-galactosidase-8D3 conjugate. This level of β-galactosidase enzyme activity in brain cannot be detected with histochemistry using colorimetric methods such as the standard X-gal technique, where a minimal enzyme activity level of 2,000 mU/g is required. It was not possible to inject even larger amounts of enzyme/MAb conjugate, because the dose used for the histochemical study in FIG. 10 is a saturating concentration of the TfRMAb. The dose of 300 μg of β-galactosidase conjugated to 600 μg of 8D3/SA per mouse is equivalent to 12 mg/kg of the β-galactosidase and 24 mg/kg of the 8D3/SA conjugate, and this dose of 8D3 TfRMAb completely saturates the BBB TfR. The BBB transport of the MAb is 50% saturated at a systemic dose of 2-4 mg/kg of the 8D3 MAb (Lee et al, 2000).

Although the β-galactosidase enzyme activity could not be detected in brain parenchyma with the histochemical method, the presence of the enzyme in the intra-endothelial compartment of brain could be detected following the intravenous administration of the high dose of the β-galactosidase-8D3 conjugate (FIGS. 10A and B). This histochemical assay demonstrates the targeting of the enzyme to the BBB compartment of brain, whereas no measurable enzyme activity was detected in the endothelial compartment following intravenous injection of the unconjugated enzyme (FIG. 10C). The histochemical product in the endothelial compartment of brain was not due to entrapment of the enzyme in the blood compartment because the brain was saline cleared prior to perfusion fixation for the histochemistry. The adequacy of the saline clearance is demonstrated by the inability to detect histochemical product in the capillary compartment following injection of the unconjugated enzyme (FIG. 10C).

It is possible to detect the β-galactosidase enzyme activity in the endothelial cell of brain because this compartment has such a small volume. The intra-endothelial compartment in brain, <1 μl/g, is about 1000-fold lower than the extra-vascular volume in brain (Pardridge, 2001). Therefore, when the enzyme-TfRMAb conjugate passes through the endothelial compartment, the enzyme activity is concentrated in the small endothelial volume, which allows for light microscopic histochemical detection. An identical intra-endothelial vascular staining pattern was reported previously following systemic administration of a TfRMAb conjugated to 5 nm gold (Bickel et al, 1994). The localization of the TfRMAb in the intra-endothelial compartment of brain was detected with light microscopy with an immunogold silver staining technique. It was not possible to detect the TfRMAb in brain parenchyma at the light microscopic level owing to the 1000-fold dilution that occurs when the antibody passes through the endothelial compartment and enters the extra-vascular compartment of brain (Bickel et al, 1994). Similarly, it is possible to detect Trojan horse mediated uptake into the brain endothelium, but not into brain parenchyma (FIG. 10).

The transport of the β-galactosidase/TfRMAb conjugated across the BBB and into brain parenchyma was demonstrated with the capillary depletion technique as shown in FIG. 11. Enzyme activity in brain homogenate was measured at 60 minutes following the intravenous injection of the β-galactosidase/8D3 conjugate. Following capillary depletion of the brain homogenate, there is a >90% removal of the capillary compartment from brain. The β-galactosidase enzyme activity in the post-vascular supernatant is >90% of the corresponding enzyme activity in the homogenate following IV injection of the β-galactosidase-TfRMAb conjugate (FIG. 11). Therefore, more than 90% of the β-galactosidase/8D3 conjugate that enters into the endothelial compartment passes through the BBB to enter brain parenchyma. This observation is in accord with prior work, which showed that >80% of the TfRMAb undergoes transcytosis through the BBB and into brain parenchyma within a 10-minute internal carotid artery perfusion of brain (Skarlatos et al, 1995).

### EXAMPLE 12

### Enzyme/Trojan Horse Fusion Proteins as Human Therapeutics

The enzyme can be conjugated to the BBB Trojan Horse with avidin-biotin technology, as shown in FIGS. 7-11, and as taught in U.S. Pat. No. 6,287,792. Alternatively, the enzyme may be fused to the molecular Trojan horse following the initial engineering of an enzyme/Trojan horse fusion gene. In the avidin-biotin approach, a fusion protein is produced with genetic engineering, whereby the avidin monomer is fused to the carboxyl terminus of the heavy chain of the Trojan horse MAb. In parallel, the enzyme is mono-biotinylated. It is important that higher degrees of biotinylation are not employed. If more than 1 biotin is attached to the enzyme, then high molecular weight aggregates may form upon mixing with the MAb/avidin fusion protein, owing to the multivalency of biotin binding by the MAb/avidin fusion protein.

Enzyme/Trojan horse fusion proteins may also be engineered without the use of avidin-biotin technology. In this approach, the cDNA encoding for the human enzyme (E) is fused to gene encoding the Trojan horse. If the Trojan horse is a MAb, comprised of a heavy chain (HC) and a light chain (LC), then the enzyme may be fused to the carboxy terminus of either the HC or LC protein; in this case the enzyme cDNA would be lacking the amino acid sequence encoding for the signal peptide. Alternatively, the enzyme could be fused to the amino terminus of either the LC or HC gene; in this case, the enzyme cDNA might include the sequence for the enzyme signal peptide, or that of another signal peptide. Alternatively, the enzyme could be fused to either the amino or carboxyl termini of a single chain Fv (ScFv) antibody, which targets a BBB receptor. Alternatively, the enzyme could be fused to the amino or carboxyl terminus of an endogenous peptide or a modified peptide that targets a BBB receptor to initiate RMT across the BBB.

If an MAb is used as the molecular Trojan horse, then standard genetic engineering techniques may be used to convert the original murine MAb to either a chimeric MAb or a humanized MAb, so that immune reactions in humans are not generated. The preferred molecular Trojan horse is a genetically engineered MAb to the human insulin receptor (HIR), designated HIRMAb. The HIRMAb is transported across the BBB up to 9-fold faster than any other Trojan horse, including TfRMAb's (Pardridge, 2001). A genetically engineered chimeric HIRMAb has been produced, and BBB transport properties of the chimeric HIRMAb at both the human BBB in vitro and at the primate BBB in vivo are comparable to the original murine HIRMAb (Coloma et al, 2000). A genetically engineered humanized HIRMAb has been produced, and BBB transport properties of the chimeric HIRMAb at both the human BBB in vitro and at the primate BBB in vivo are comparable to the original murine HIRMAb (Pardridge and Boado published U.S. patent application 2004/0101904A1).

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the above preferred embodiments and examples, but is only limited by the following claims.

### BIBLIOGRAPHY

1. Arghi Conn., Hartnell L M, Aguilar R C, Haft C R, Bonifacino J S. 2004. Role of mammalian retromer in sorting of the cation-independent mannose 6-phosphate receptor. J. Cell Biol. 165: 123-133.
2. Beisiegel, U., et al (1981) Monoclonal antibodies to the low density lipoprotein receptor as probes for study of receptor-mediated endocytosis and the genetics of familial hypercholesterolemia. J. Biol. Chem. 256: 11923-11931.
3. Bickel, U, Kang Y S, Yoshikawa T and Pardridge W M (1994) In vivo demonstration of subcellular localization of anti-transferrin receptor monoclonal antibody-colloidal gold conjugate in brain capillary endothelium. J Histochem Cytochem 42: 1493-1497.
4. Boado R J, Li J Y, Nagaya M, Zhang C, Pardridge W M. 1999. Selective expression of the large neutral amino acid transporter in the blood-brain barrier. Proc Natl Acad Sci USA. 96: 12079-12084.
5. Bruggemann, M. et al. (1989) “The immunogenicity of chimeric antibodies,” J. Exp. Med., 170: 2153-2157.
6. Cheng S H and Smith A E (2003) Gene therapy progress and prospects: gene therapy of lysosomal storage disorders. Gene Ther 10: 1275-1281.
7. Cheng, S. H. and Smith, A. E. (2003): Gene therapy progress and prospects: gene therapy of lysosomal storage disorders. Gene. Ther., 10: 1275-1281.
8. Coloma, M. J., Lee, H. J., Kurihara, A., Landaw, E. M., Boado, R. J., Morrison, S. L., and Pardridge, W. M. (2000) “Transport across the primate blood-brain barrier of a genetically engineered chimeric monoclonal antibody to the human insulin receptor,” Pharm. Res., 17: 266-274.
9. Coloma, M. J., Hastings, A., Wims, L. A., and Morrison, S. L. (1992) “Novel vectors for the expression of antibody molecules using variable regions generated by polymerase chain reaction,” J. Immunol. Methods, 152: 89-104.
10. Datla, R. S., Hammerlindl, J. K., Pelcher, L. E., Crosby, W. L. and Selvaraj, G. (1991): A bifunctional fusion between beta-glucuronidase and neomycin phosphotransferase: a broad-spectrum marker enzyme for plants. Gene, 101: 239-246.
11. Domingo, D. L. and Trowbridge, I. S. (1985): Transferrin receptor as a target for antibody-drug conjugates. Methods Enzymology 112: 238-247.
12. Foote, J. and Winter, G. (1992) “Antibody framework residues affecting the conformation of the hypervariable loops,” J. Mol. Biol., 224: 487-499.
13. Gehrmann, M. C., Opper, M., Sedlacek, H. H., Bosslet, K. and Czech, J. (1994): Biochemical properties of recombinant human beta-glucuronidase synthesized in baby hamster kidney cells. Biochem. J., 301: 821-828.

14. Graziano, R. F. et al. (1995) “Construction and characterization of a humanized anti-γ-Ig receptor type I (FcγRI) monoclonal antibody,” J. Immunol., 155: 4996-5002.
15. Green, N. M. (1975): Avidin. Adv. Protein Chem., 29: 85-133.
16. Haisma, J. J. et al (2000) Construction and characterization of a fusion protein of single-chain anti-CD20 antibody and human □-glucuronidase for antibody-directed enzyme prodrug therapy. Blood 92: 184-190.
17. Juers D H, et al. 2000. High resolution refinement of beta-galactosidase in anew crystal form reveals multiple metal-binding sites and provides a structural basis for alpha-complementation. Protein Science. 9: 1685-1699.
18. Kakkis E, et al. 2004. Intrathecal enzyme replacement therapy reduces lysosomal storage in the brain and meninges of the canine model of MPS I. Mol Genet Metab. 83: 163-174.
19. Kaye E M. 2001. Lysosomal storage diseases. Curr Treat Options Neurol. 3: 249-256.
20. Lee H J, Engelhardt B, Lesley J, Bickel U and Pardridge W M (2000) Targeting rat anti-mouse transferrin receptor monoclonal antibodies through blood-brain barrier in mouse. J Pharmacol Exp Ther 292: 1048-1052.
21. Li, J. Y., Sugimura, K., Boado, R. J., Lee, H. J., Zhang, C., Dubel, S., and Pardridge, W. M. (1999) “Genetically engineered brain drug delivery vectors—cloning, expression, and in vivo application of an anti-transferrin receptor single chain antibody-streptavidin fusion gene and protein,” Protein Engineering, 12: 787-796.
22. Miller, G. (2002) “Breaking down barriers,” Science, 297: 1116-1118.
23. Pardridge W M (2001) Brain Drug Targeting; The Future of Brain Drug Development. Cambridge University Press, Cambridge, United Kingdom.
24. Pardridge, W. M. (1997) “Drug delivery to the brain,” J. Cereb. Blood Flow. Metabol., 17: 713-731.
25. Pardridge W M, Boado R J, Farrell C R. 1990. Brain-type glucose transporter (GLUT-1) is selectively localized in the blood-brain barrier. Studies with quantitative western blotting and in situ hybridization. J Biol. Chem. 265: 18035-18040.
26. Pardridge, W. M., Buciak, J. L., and Friden, P. M. (1991) “Selective transport of anti-transferrin receptor antibody through the blood-brain barrier in vivo,” J. Pharmacol. Exp. Ther., 259: 66-70.
27. Pardridge, W. M., Kang, Y.-S., Buciak, J. L., and Yang, J. (1995) “Human insulin receptor monoclonal antibody undergoes high affinity binding to human brain capillaries in vitro and rapid transcytosis through the blood-brain barrier in vivo in
28. the primate,” Pharm., Res. 12: 807-816.
29. Pardridge W M, Buciak J L, Yoshikawa T. 1992. Transport of recombinant CD4 through the rat blood-brain barrier in vivo. J Pharmacol Exp Ther. 261: 1175-1180.
30. Pardridge W M, Eisenberg J, Yang J. 1985. Human blood-brain barrier insulin receptor. J Neurochem. 44: 1771-1778.
31. Pardridge W M, Eisenberg J, Yang J. 1987. Human blood-brain barrier transferring receptor. Metabolism. 36: 892-985.
32. Pardridge W M. 2002. Drug and gene delivery to the brain: the vascular route. Neuron. 36: 555-558.
33. Pichla, W. L., Murali, R., and Burnett, R. M. (1997) “The crystal structure of a Fab fragment to the melanoma-associated GD2 ganglioside,” J. Struct. Biol., 119: 6-16.
34. Rechavi, G. et al (1983) “Evolutionary aspects of immunoglobulin heavy chain variable region (VH) gene subgroups”. Proc. Natl. Acad. Sci. (U.S.A.) 80: 855-859.
35. Schechter, Y., Maron, R. and Cohen I. R. (1982) Autoantibodies to insulin receptor spontaneously develop as anti-idiotypes in mice immunized with insulin. Science, 216: 542-545.
36. Schier, R. et al. (1996) “Isolation of picomolar affinity anti-c-erbB-2 single-chain Fv by molecular evolution of the complementarity determining regions in the center of the antibody binding site,” J. Mol. Biol., 263: 551-557.
37. Shin, S. U. and Morrison, S. L. (1990) “Expression and characterization of an antibody binding specificity joined to insulin-like growth factor 1: potential applications for cellular targeting”. Proc. Natl. Acad. Sci. U.S.A. 87: 5322-5326.
38. Skarlatos S, Yoshikawa T and Pardridge W M (1995) Transport of [125I]transferrin through the rat blood-brain barrier. Brain Res 683: 164-171.
39. Soos, M. A., et al (1986) “Monoclonal antibodies reacting with multiple epitopes on the human insulin receptor”. Biochem. J. 235: 199-208.
40. Triguero D, Buciak J and Pardridge W M (1990) Capillary depletion method for quantification of blood-brain barrier transport of circulating peptides and plasma proteins. J Neurochem 54: 1882-1888.
41. U.S. Pat. No. 5,624,659 (issued Apr. 29, 1997) “Method of Treating Brain Tumors Expressing Tenascin” (Inventors: Darell D. Bigner and Michael R. Zalutsky; Assignee: Duke University).
42. U.S. Pat. No. 6,287,792 (issued Sep. 11, 2001) “Drug delivery of antisense oligonucleotides and peptides to tissues in vivo and to cells using avidin-biotin technology (Inventors: William M. Pardridge and Ruben J. Boado; Assignee: University of California).
43. Vogel, C. L. et al. (2002) “Efficacy and safety of trastuzumab as a single agent in first-line treatment of HER2-overexpressing metastatic breast cancer,” J. Clin. Oncol., 20: 719-726.
44. Wu D, Pardridge W M. 1996. Central nervous system pharmacologic effect in conscious rats after intravenous injection of a biotinylated vasoactive intestinal peptide analog coupled to a blood-brain barrier drug delivery system. J Pharmacol Exp Ther. 279: 77-83.
45. Wu, D., Yang, J., and Pardridge, W. M. (1997): “Drug targeting of a peptide radiopharmaceutical through the primate blood-brain barrier in vivo with a monoclonal antibody to the human insulin receptor.” J. Clin. Invest., 100: 1804-1812.
46. Zhang, Y., Lee, H. J., Boado, R. J., and Pardridge, W. M. (2002) “Receptor-mediated delivery of an antisense gene to human brain cancer cells,” J Gene Med., 4:183-194.

1. A composition that is capable of delivering a large enzyme across the blood brain barrier, said composition comprising:
a large enzyme; and
a blood-brain barrier targeting agent wherein said blood brain barrier targeting agent is linked to said large enzyme.

2. A composition according to claim 1 wherein said blood brain barrier targeting agent is selected from the group consisting of transferrin, insulin, leptin, insulin-like growth factors, cationic peptides, lectins, peptidomimetic monoclonal antibodies to the transferrin receptor, peptidomimetic monoclonal antibodies to the insulin receptor, peptidomimetic monoclonal antibodies to the insulin-like growth factor receptor, and peptidomimetic monoclonal antibodies to the leptin receptor.

3. A composition according to claim 1 wherein said large enzyme is a lysosomal enzyme.

4. A composition according to claim 2 wherein said large enzyme is a lysosomal enzyme.

5. A composition according to claim 1 wherein said large enzyme is biotinylated and said blood brain barrier targeting agent comprises avidin or streptavidin and wherein said large enzyme is linked to said blood brain barrier targeting agent via at least one avidin-biotin linkage.

6. A composition according to claim 5 wherein said large enzyme is monobiotinylated.

7. A composition according to claim 1 wherein said blood brain barrier targeting agent is linked to said large enzyme by genetic fusion to form a fusion protein consisting essentially of said blood brain barrier targeting agent and said large enzyme.

8. A pharmaceutical preparation for intravenous administration, said pharmaceutical preparation comprising a composition according to claim 1 and an acceptable carrier for said composition to provide for intravenous administration of said pharmaceutical preparation.

9. A pharmaceutical preparation according to claim 8 wherein said blood brain barrier targeting agent is selected from the group consisting of transferrin, insulin, leptin, insulin-like growth factors, cationic peptides, lectins, peptidomimetic monoclonal antibodies to the transferrin receptor, peptidomimetic monoclonal antibodies to the insulin receptor, peptidomimetic monoclonal antibodies to the insulin-like growth factor receptor, and peptidomimetic monoclonal antibodies to the leptin receptor.

10. A composition according to claim 8 wherein said large enzyme is a lysosomal enzyme.

11. A composition according to claim 9 wherein said large enzyme is a lysosomal enzyme.

12. A method for increasing the ability of a large enzyme to cross the human blood brain barrier comprising the step of linking said large enzyme to a blood brain barrier targeting agent.

13. A method according to claim 12 wherein said blood brain barrier targeting agent is selected from the group consisting of transferrin, insulin, leptin, insulin-like growth factors, cationic peptides, lectins, peptidomimetic monoclonal antibodies to the transferrin receptor, peptidomimetic monoclonal antibodies to the insulin receptor, peptidomimetic monoclonal antibodies to the insulin-like growth factor receptor, and peptidomimetic monoclonal antibodies to the leptin receptor.

14. A method according to claim 12 wherein said large enzyme is a lysosomal enzyme.

15. A method according to claim 13 wherein said large enzyme is a lysosomal enzyme.

16. A method according to claim 12 wherein said large enzyme is linked to said blood brain barrier targeting agent via an avidin-biotin linkage.

17. A method according to claim 12 wherein said enzyme is linked to said blood brain barrier targeting agent by genetic fusion.

18. A method for intravenously administering a lysosomal enzyme to a human patient to provide enzyme replacement therapy to said human patient, said method comprising the step of injecting a pharmaceutical preparation according to claim 8 into the blood stream of said human patient.

19. A method for intravenously administering a lysosomal enzyme to a human patient to provide enzyme replacement therapy to said human patient, said method comprising the step of injecting a pharmaceutical preparation according to claim 9 into the blood stream of said human patient.

20. A method for intravenously administering a lysosomal enzyme to a human patient to provide enzyme replacement therapy to said human patient, said method comprising the step of injecting a pharmaceutical preparation according to claim 10 into the blood stream of said human patient.

21. A method for intravenously administering a lysosomal enzyme to a human patient to provide enzyme replacement therapy to said human patient, said method comprising the step of injecting a pharmaceutical preparation according to claim 11 into the blood stream of said human patient.
